(19) United States
(12) Patent Application Publication
(10) Pub. No.: US 2018/0096104 A1
(43) Pub. Date: Apr. 5, 2018

(54) DISEASE MANAGEMENT SYSTEM

(71) Applicant: MED Inc., Richmond, VA (US)

(72) Inventors: Shane M. MALEY, Richmond, VA (US); David OGENS, Charlottesville, VA (US); Shawn M. HARGER, Richmond, VA (US)

(21) Appl. No.: 15/726,277

(22) Filed: Oct. 5, 2017

Related U.S. Application Data
(60) Provisional application No. 62/404,665, filed on Oct. 5, 2016.

(51) Int. Cl. G06F 19/00; G06F 17/30

(52) U.S. Cl. CPC G06F 19/322; G06F 19/3431; G06F 19/326; G06F 17/30867; G06F 19/363

(57) ABSTRACT

A system, a method, and a computer program for providing COPD and similar management service are disclosed. The system may include three main branches: patients, users and a service provider. The service provider provides management services of the COPD patients to the users.

### CROSS-REFERENCE TO RELATED APPLICATIONS

This application claims benefit and priority to U.S. Provisional Application No. 62/404,665, filed Oct. 5, 2016, the disclosure of which is incorporated by reference herein in its entirety.

### FIELD OF THE DISCLOSURE

The present disclosure relates to a network system, a network method, and a computer program for communicating a display signal to a device on a network; and, more particularly, communicating with a sensor at a remote location, managing a database record associated with the sensor, and generating and communicating the display signal to the device on the network.

### BACKGROUND OF THE DISCLOSURE

Chronic diseases in humans usually start slowly and get worse over time. Many factors can play a role in a disease's progression. Examples of chronic diseases include, for example, Alzheimer's disease, chronic obstructive pulmonary, asthma, and the like.

Chronic obstructive pulmonary disease (COPD), for example, is a type of obstructive lung disease characterized by long-term poor airflow. Chronic bronchitis and emphysema are older terms used for different types of COPD. The main symptoms include shortness of breath and cough with sputum production. COPD typically worsens over time, and patients eventually find walking up or down stairs or carrying things difficult. Tobacco smoking is the most common cause of COPD, with a number of other factors such as air pollution and genetics playing a smaller role. In the developing world, one of the common sources of air pollution is poorly vented heating and cooking fires. Long-term exposure to these irritants causes an inflammatory response in the lungs resulting in narrowing of the small airways and breakdown of lung tissue. The diagnosis is based on poor airflow as measured by lung function tests. In contrast to asthma, the airflow reduction does not improve much with the use of a bronchodilator.

As of 2013, COPD is estimated to affect over 16 million people in the U.S. or nearly 5 percent of the population. It typically occurs in people over the age of 40, and both males and females are affected equally. In 2013, COPD resulted in 2.9 million deaths, up from 2.4 million deaths in 1990. It is the third leading cause of death in the U.S. More than $32 billion was spent on COPD-related care in 2010. There is no known cure for COPD, but the symptoms are treatable and its progression can be delayed. The major goals of management are to reduce risk factors, to manage stable COPD, to prevent and treat acute exacerbations, and to manage associated illnesses. The only measures that have been shown to reduce mortality are smoking cessation and supplemental oxygen. Stopping smoking decreases the risk of death by 18%. Other recommendations include influenza vaccination once a year, pneumococcal vaccination once every 5 years, and reduction in exposure to environmental air pollution. In those with advanced disease, palliative care may reduce symptoms, with morphine improving the feelings of shortness of breath. Noninvasive ventilation may be used to support breathing.

Currently, healthcare providers such as hospitals experience high admission rates for patients with severe respiratory issues related to COPD or neuromuscular diseases, many of which may be avoidable. An unfulfilled need exists for a system, method and computer program to stage patients by severity of illness along with key triggers that influence exacerbations.

### SUMMARY OF THE DISCLOSURE

According to an aspect of the disclosure, a network system, a network method and a computer program are disclosed for communicating over a network with a sensor at a remote location, managing a database record associated with the sensor, including staging the record by severity of illness of the patient along with one or more key triggers that may influence exacerbations, and generating and communicating a display signal to a device on the network to verify patient severity of illness and create one or more pathways to prevent exacerbations.

In one aspect, a network system for managing patient care includes a server operatively in communication with at least one health sensor over a network, the health sensor assigned to at least one patient, the server configured to receive at least one message from the health sensor containing data associated with sensed patient data, a database coupled to the server for storing the sensed patient data and displaying on a display device by the server a status of at least one health condition of the patient according to a predetermined rating and based on a history of the at least one received messages stored in the database for managing treatment of the at least one patient. The at least one health sensor may measure oxygen levels or carbon dioxide levels in the at least one patient. The at least one message may comprise a plurality of messages. The health condition may be a Chronic Obstructive Pulmonary Disease (COPD) or a neuromuscular disorder. The server may be configured to display a list of patients related to COPD or the neuromuscular disorder. The server may be configured to display a historical summary of patient visits including at least one color coded visual indicator associated with each displayed patient visit, the at least one color coded visual indicator indicative of a specific health characteristic of the patient. The server may be configured to display a summary page of an individual COPD patient that includes a plurality of, in any combination: an initial CCQ Score, a trending SpO2 Level, a current ETCO2 Level, a currently smoking Indicator, a CCQ score, a current SpO2 level, a device compliance indicator, a weight change indicator, a trending ETCO2 Level, a respiratory medication compliance indicator, a respiratory therapist assigned to the patient and a separate visual color coded indicator indicative of a specific health characteristic of the COPD patient. Any indicator or level is itself color coded to indicate a level of health or compliance. Moreover, the server may be configured to display a summary page of an individual neuromuscular patient that includes a plurality of, in any combination: trending vital capacity sitting up indicator, a smoking indicator, a trending SpO2 level indicator, a current vital capacity sitting up indicator, a weight change indicator, a device compliance indicator, a current SpO2 level, a medical compliance indicator, a current EtCO2 Level, a respiratory therapist and a separate color coded visual indicator indicating a specific health characteristic of the patient. Any indicator or level is itself color coded to indicate a level of health or compliance. The server may also be configured to display a patient intake form and receiving input date for adding a new patient to the network system. The server may be configured to display a patient evaluation form for inputting results of a physical evaluation and a mental evaluation. The server may be configured to display a CCQ questionnaire for receiving input. The server may be configured to display a device management screen to enter information relate to a new device including a health sensor into system inventory, remove a device from system inventory, or to assign a device to a patient, the information may include a serial number, model and manufacturer. The server may be configured to display a medication and vaccine screen for adding, updating or removing a medication or vaccine in the system, or for noting potential medication variances from standard practice. The server may be configured to display a COPD interactive patient dashboard that includes an indication of all or a portion of patients managed by the system by individual Global Initiative for Obstructive Lung Disease (GOLD) stages; the indication may be a color coded pie chart. The server may be configured to display a neuromuscular Interactive patient dashboard that may include an indication of all or a portion of patients managed by the system by vital capacity or ventilation usage; the indication may be a color coded pie chart.

In one aspect, method for managing patient care includes receiving at a server over a network from at least one health sensor assigned to at least one patient, at least one message from the health sensor containing data associated with sensed patient data, capturing the sensed patient data and storing in a database and displaying on a display device by the server a status of at least one health condition of the patient according to a predetermined rating based on a history of the at least one received messages stored in the database for managing treatment of the at least one patient. The at least one health sensor may measure oxygen levels or carbon dioxide levels in the at least one patient. The at least one message may be a plurality of messages. The health condition is a Chronic Obstructive Pulmonary Disease (COPD) or a neuromuscular disorder. The method may further include displaying a summary page of an individual COPD patient that includes a plurality of, in any combination: an initial CCQ Score, a trending SpO2 Level, a current ETCO2 Level, a currently smoking Indicator, a CCQ score, a current SpO2 level, a device compliance indicator, a weight change indicator, a trending ETCO2 Level, a respiratory medication compliance indicator, a respiratory therapist assigned to the patient and a separate visual color coded indicator indicative of a general current overall health of the COPD patient, wherein any indicator or level may itself be color coded to indicate a level of health or compliance. The method may further include displaying a summary page of an individual neuromuscular patient that includes a plurality of, in any combination: trending vital capacity sitting up indicator, a smoking indicator, a trending SpO2 level indicator, a current vital capacity sitting up indicator, a weight change indicator, a device compliance indicator, a current SpO2 level, a medical compliance indicator, a current EtCO2 Level, a respiratory therapist, and a separate color coded visual indicator indicating a general overall health of the patient, wherein any indicator or level may itself be color coded to indicate a level of health or compliance.

In one aspect, a computer program product embodied on a non-transitory computer-readable medium that comprises computer logic that when read and executed by a computer performs the following: receiving at a server over a network from at least one health sensor, including an oxygen sensor or carbon dioxide sensor, assigned to at least one patient, a plurality of messages from the health sensor containing data associated with sensed patient data, capturing the sensed patient data and storing in a database and displaying on a display device by the server a status of at least one health condition including a Chronic Obstructive Pulmonary Disease (COPD) or a neuromuscular disorder of the patient according to a predetermined rating based on a history of the plurality of received messages stored in the database for managing treatment of the at least one patient. All patient data stored in the database, or data produced and displayable by the server, may be searchable and reportable. The server may provide for a tool for evaluating Respiratory Therapist performance, quality and consistency based on metrics related to data stored in the database.

Additional features, advantages, and embodiments of the disclosure may be set forth or apparent from consideration of the detailed description and drawings. Moreover, it is to be understood that the foregoing summary of the disclosure and the following detailed description and drawings are exemplary and intended to provide further explanation without limiting the scope of the disclosure.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 shows an example of a network system, which is constructed according to the principles of the disclosure;

FIG. 2 shows an example of components that may be included in a server of the network system shown in FIG. 1, constructed according to the principles of the disclosure;

FIG. 3 shows an example of a process that may be carried out by the network server in FIG. 2, according to the principles of the disclosure;

FIG. 4A is an example of individual patient records that may be generated, according to the principles of the disclosure;

FIG. 4B is an example of specific individual patient record, according to the principles of the disclosure;

FIGS. 4C and 4D are example visit summary pages displayed when a particular time of visit record is selected from FIG. 4B;

FIG. 4E is an example of a new patient intake screen, configured according to principles of the disclosure;

FIG. 4F is an example of an evaluation record of a patient, configured according to principles of the disclosure;

FIG. 4G is an example of a CCQ questionnaire of a patient, configured according to principles of the disclosure;

FIG. 4H is an example of a device assignment, configured according to principles of the disclosure;

FIG. 4I is an example of a medications and vaccine tracking page for a patient, configured according to principles of the disclosure;

FIG. 4J is an example of an exacerbations and hospitalization history for a patient, configured according to principles of the disclosure;

FIG. 5A is an interactive administrative dashboard (IPD) display that shows the status of all COPD patients, configured according to principles of the disclosure;

FIG. 5B is an IPD display that shows the status of all neuromuscular patients, configured according to principles of the disclosure;

FIG. 6A is an example of an interactive administrative dashboard display, configured according to principles of the disclosure;

FIG. 6B is an example of an IPD display for managing patients, configured according to principles of the disclosure;

FIG. 6C is an example of an IPD display for managing devices, configured according to principles of the disclosure;

FIG. 6D is an example of an IPD display for managing medications, configured according to principles of the disclosure;

FIG. 6E is an example of an IPD display for generating reports, configured according to principles of the disclosure; and

FIG. 6F is an example of an IPD display for managing programs of related patients, configured according to principles of the disclosure.

The present disclosure is further described in the detailed description that follows.

### DETAILED DESCRIPTION OF THE DISCLOSURE

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

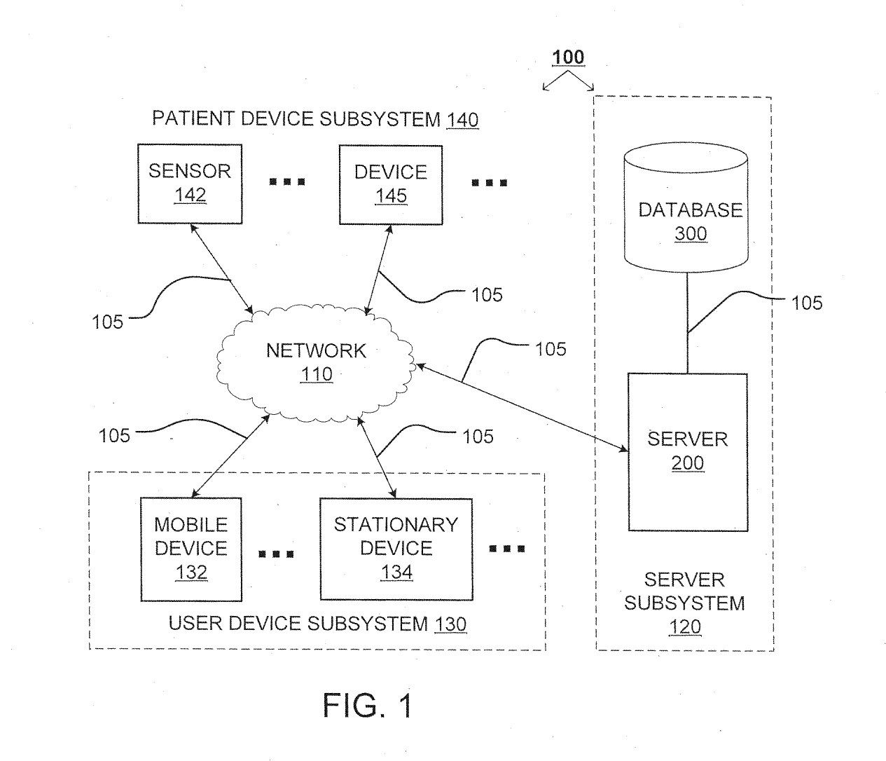

FIG. 1 shows an example of a network system 100, constructed according to the principles of the disclosure. The system 100 may include a plurality of stationary and/or mobile devices that are capable of communicating with each other via one or more networks 110, one or more communication links 105, and/or the like. The system 100 may include three subsystems, including: a server subsystem 120, a user device subsystem 130, and a patient device subsystem 140.

The patient device subsystem 140 may include one or more sensor devices 142 (e.g., heart rate sensor, oxygen level sensor, blood pressure sensor, body temperature sensor, weight sensor, carbon dioxide sensor, fitness tracker, step counter, activity sensor, vital capacity sensor, or the like) that may detect, monitor and communicate patient condition data (e.g., heart rate data, blood-oxygen level data, blood pressure data, body temperature data, body weight data, steps walked data, duration of activity data, intensity of activity data, and the like). The patient subsystem 140 may include one or more devices 145 (e.g., a computer, a smart device, a smart phone, a tablet, a smart watch, a pill-dispenser, a non-invasive inhaler, or the like) that may also include one or more sensors therein, which may be optionally deployed. Each sensor 142 and device 145 in the patient subsystem 140 may be associated with a particular patient database record in the server subsystem 120 (discussed below). The patient database record may include patient data such as, for example, a patient number, a name, an address, a telephone number, healthcare provider information, insurer name, insurer coverage, insurer policy number, current medical conditions, medical history, allergy data, prescription data, IP address, media access control (MAC) address, login identification (ID), password, and the like. All patient data stored in the database including visits and treatments and assigned device and medications is searchable and reportable. The sensor devices 142 may be a plurality of sensors associated with one patient, or different sensors 142 associated with different patients, and the sensors may provide sensed data related to the respective patient over a time period, e.g., daily, weekly, when a therapists visits a patient, or when remotely accessed from a health provider device, or combinations thereof. The time period may be programmable (e.g., locally or remotely), or predetermined when assigned. The messages from the sensors may also be sent upon a detected change in patient condition.

The patient database record, including the patient data, is associated with a particular patient. The one or more sensor devices 142 may be operatively coupled to a patient, as assigned for reading current patient data (e.g., heart rate, oxygen level, blood pressure, body temperature, weight, carbon dioxide) based on the type of sensor or sensors, and for communicating the read patient data to server 200.

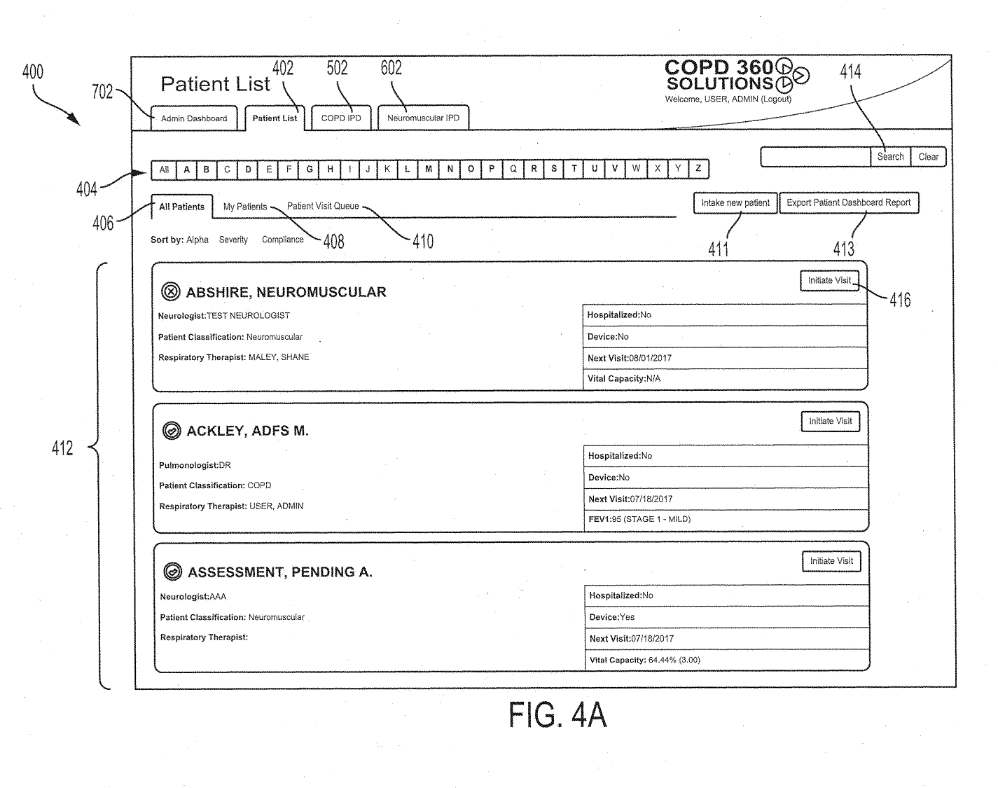

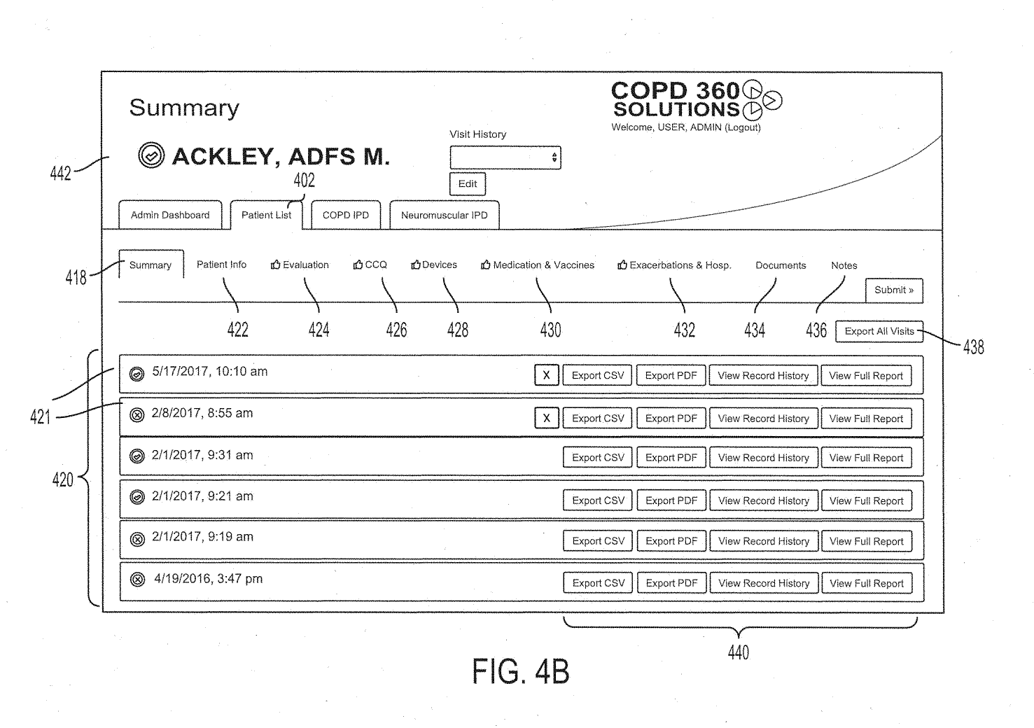

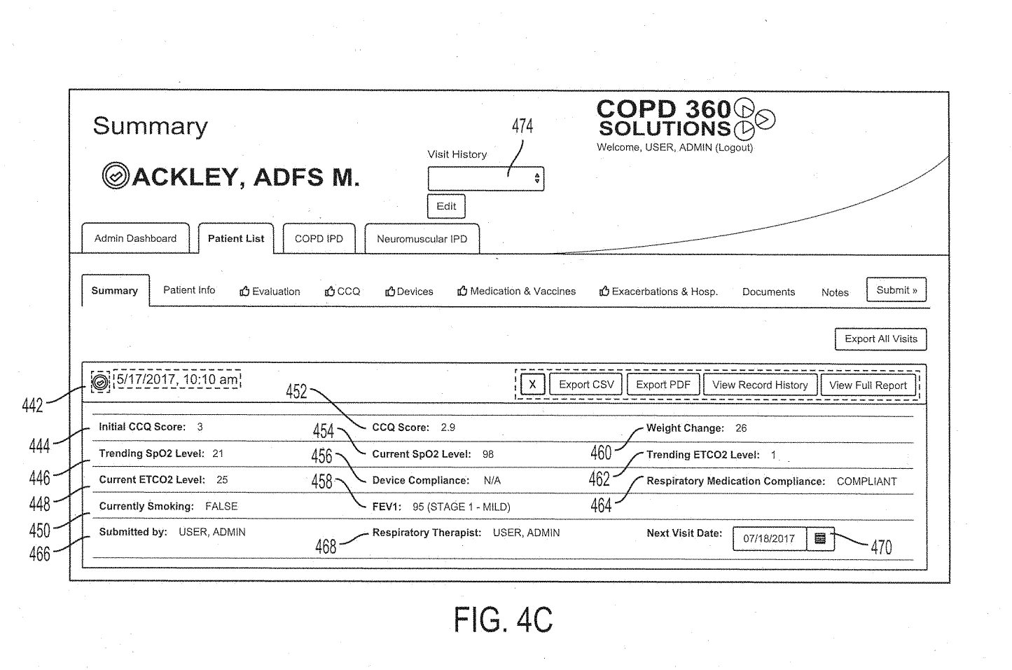

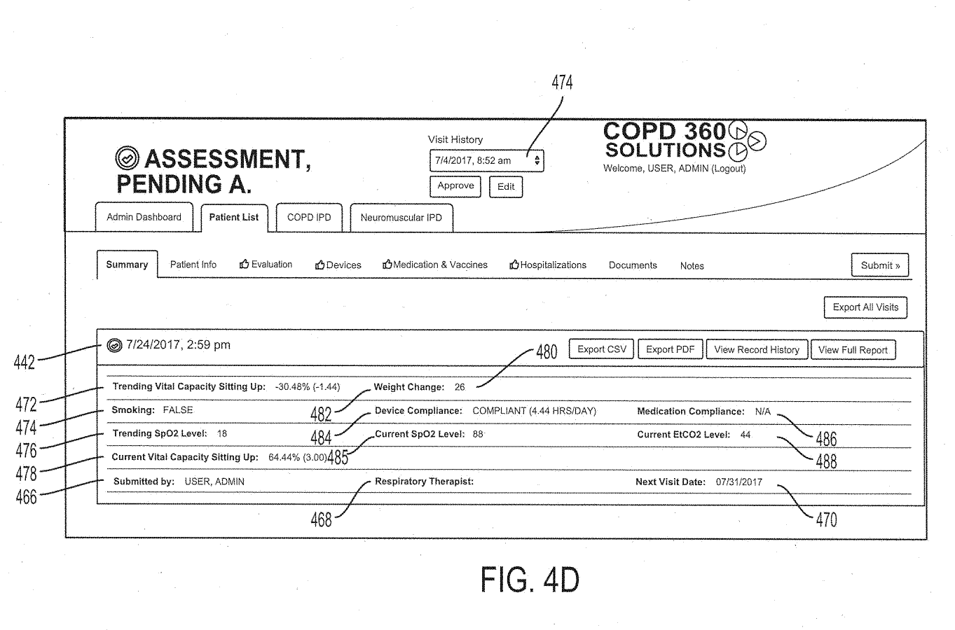

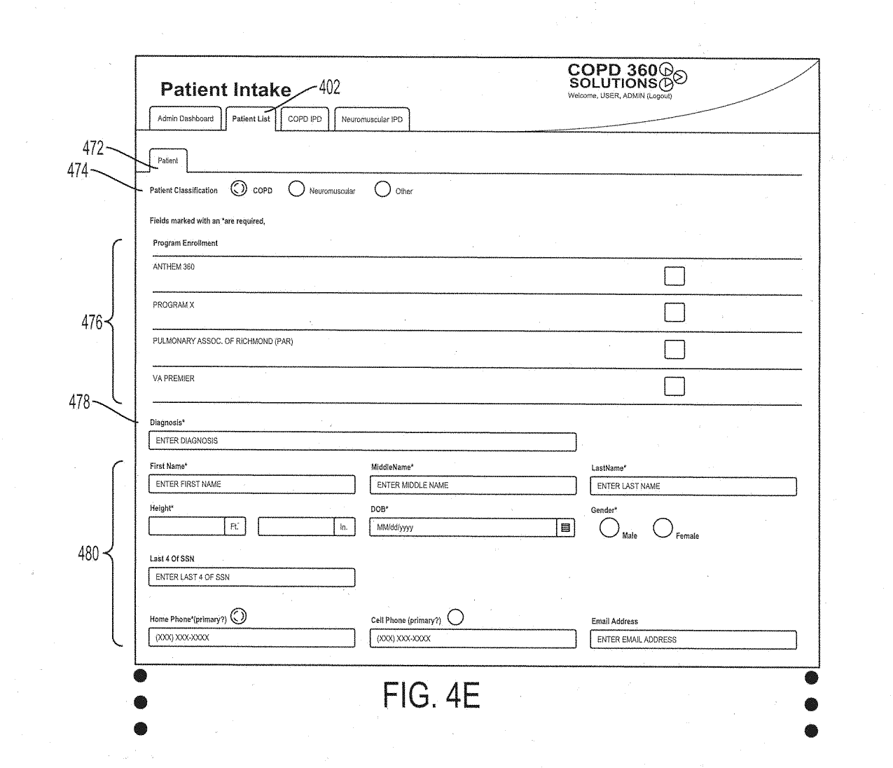

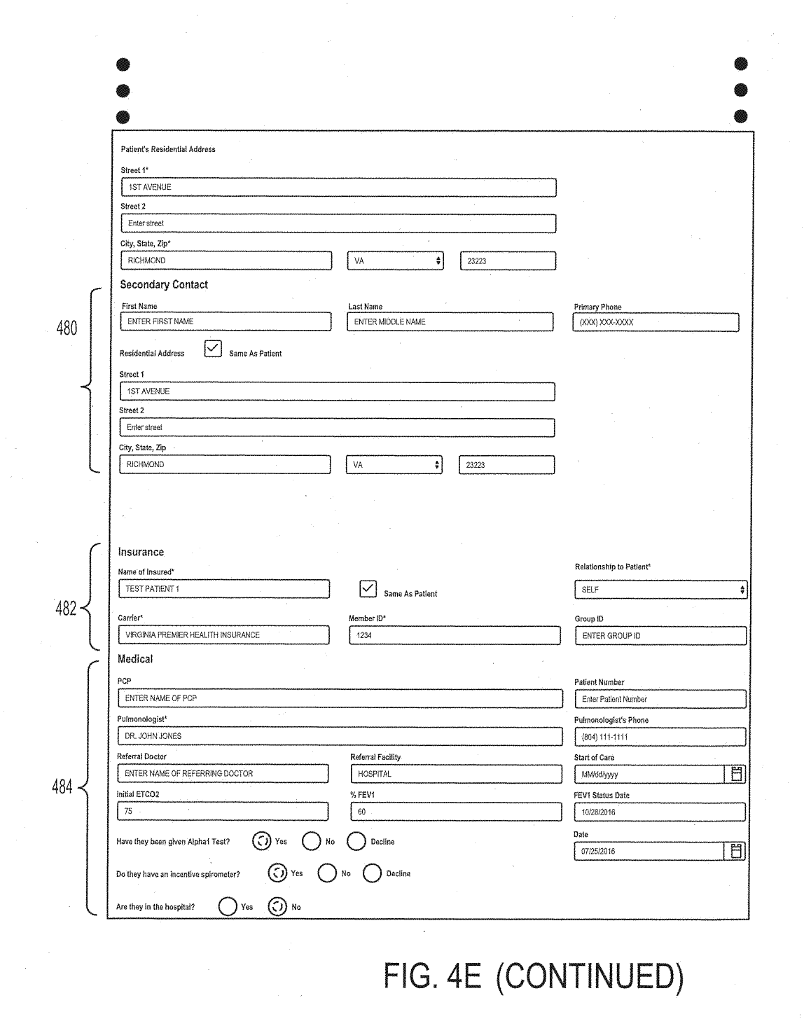

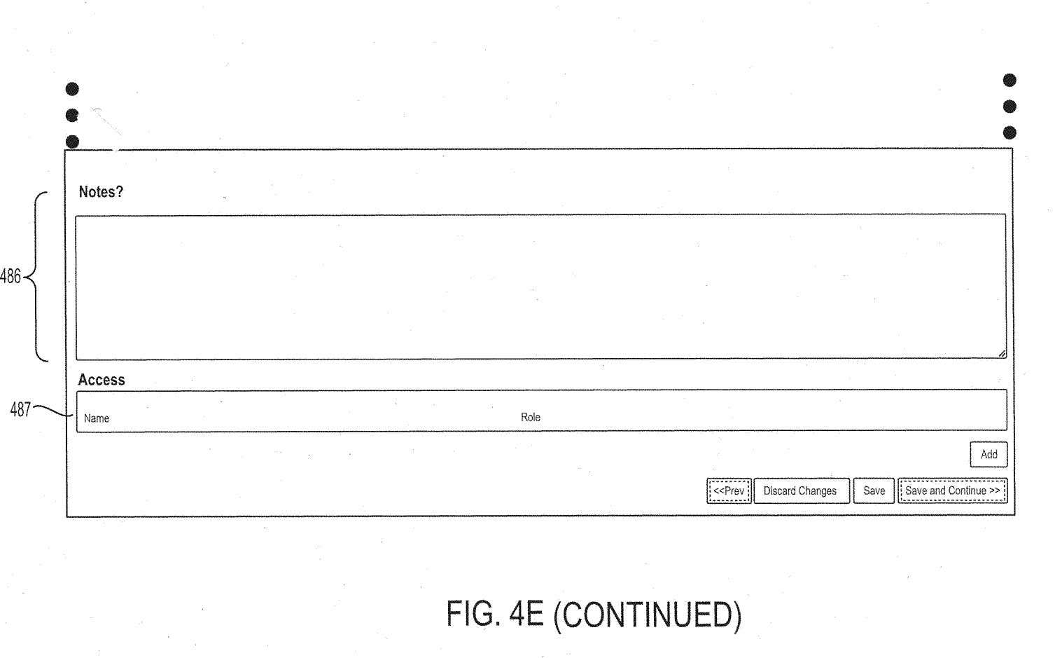

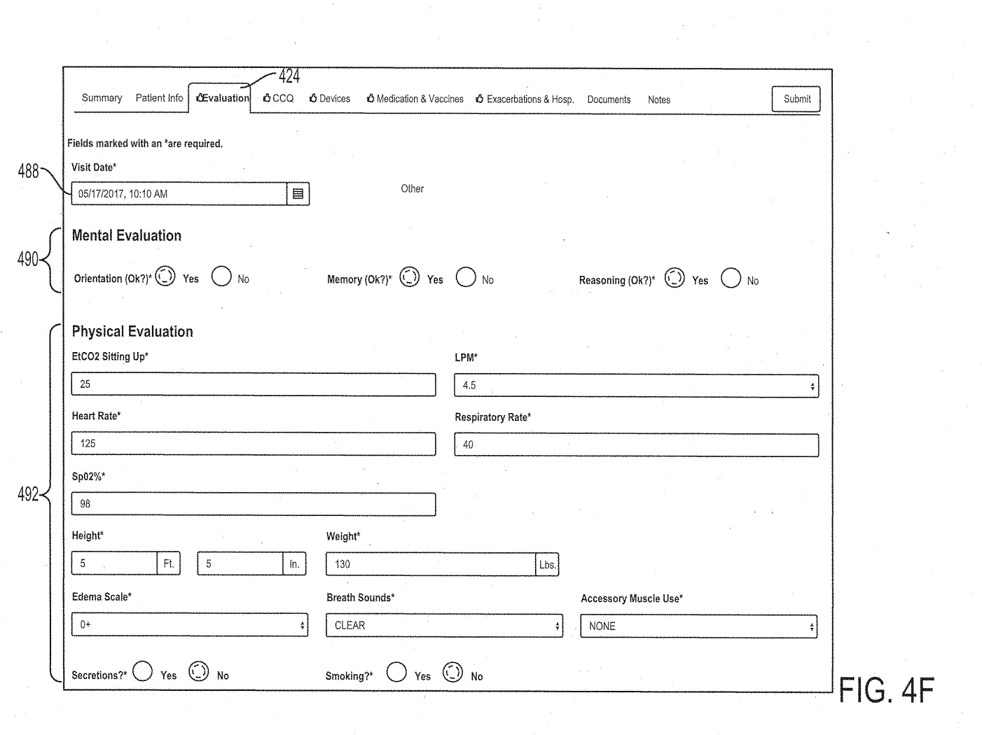

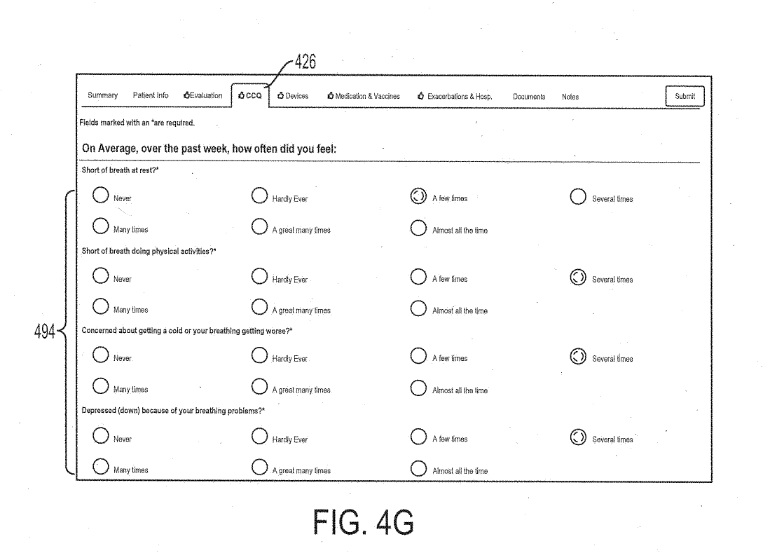

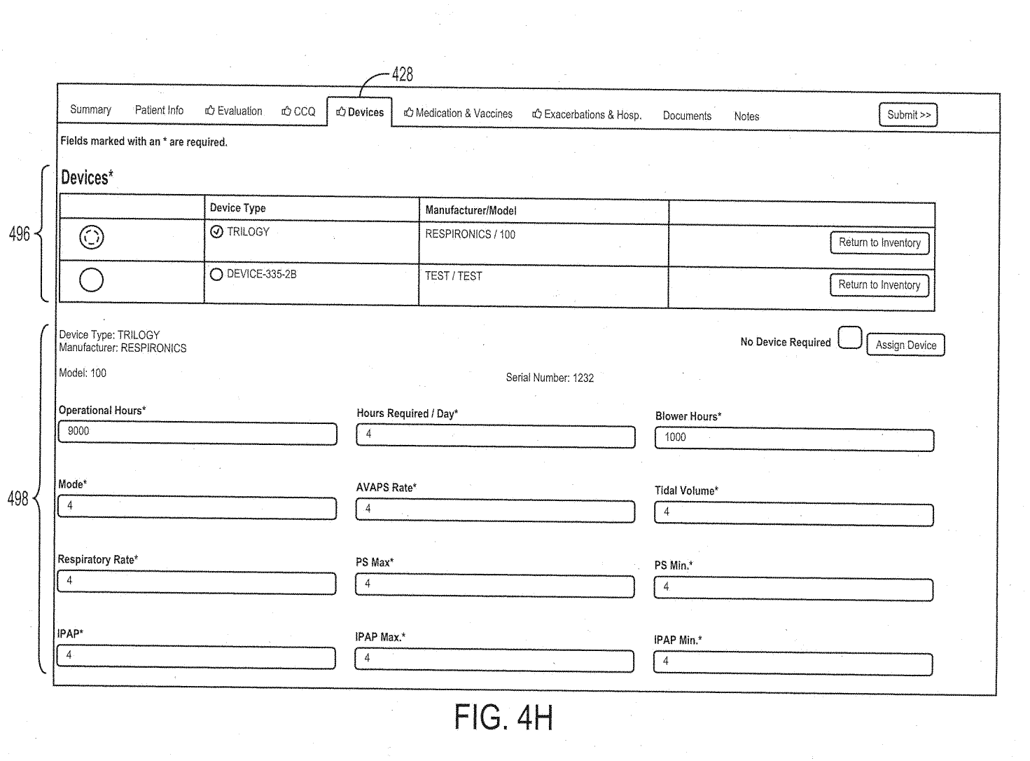

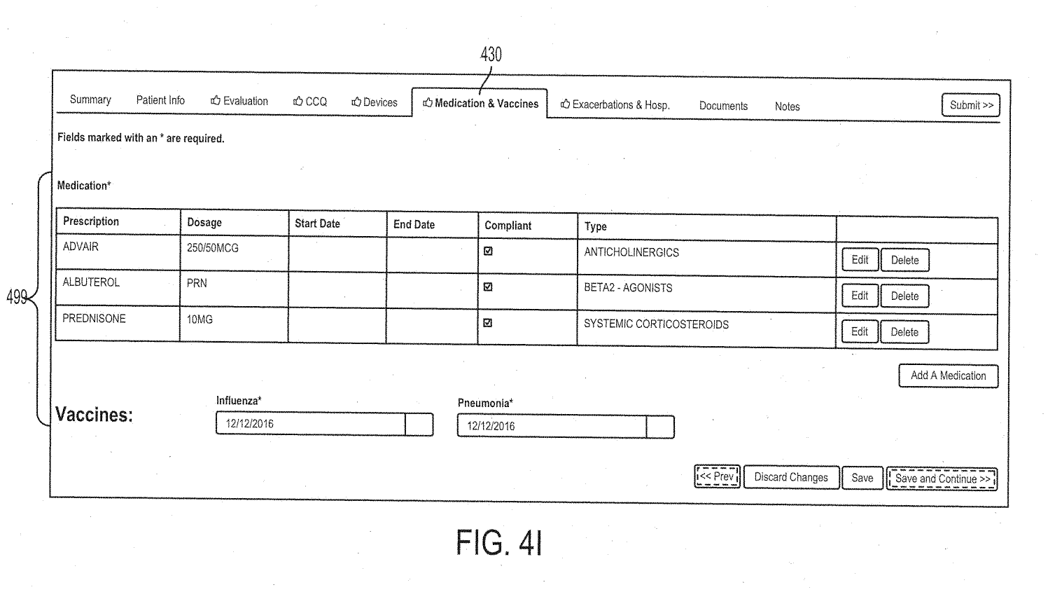

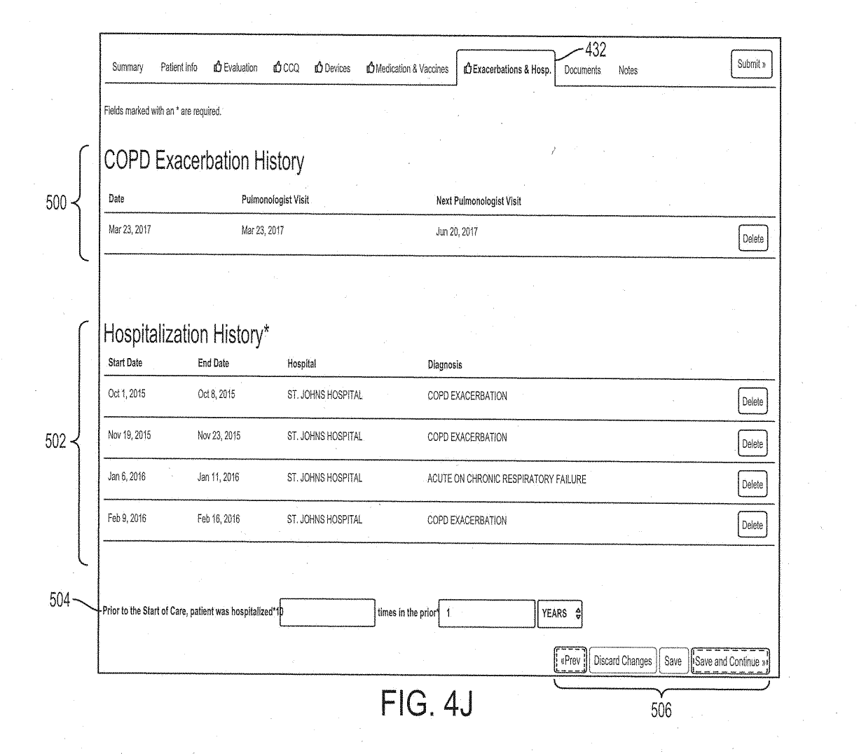

The system 100 may provide a portal for COPD and neuromuscular care providers to assess a patient to provide a specialist to deliver a home visit via mobile device 132 or stationary device 134 to easily input data related to the home visit and patient in a few entries such as on a screen or keyboard. The system 100 may provide for keeping track of devices such as sensors 142 issued to patients, while making sure no sensor 142 is improperly used. The system 100 may also track prescriptions and hospitalizations so each specialist knows, via mobile device 132 or device 134, a medical history of a patient for delivering a high level of care. The system 100 may provide reports and an interactive patient dashboard, such as shown in relation to one or more FIGS. 4A-6F, for tracking the continuing health of every patient managed in the system 100 and for regular visits that are scheduled and met.

The following description is provided with respect to patients with COPD, with the understanding that the network system 100 may be implemented similarly with patients having other types of chronic illnesses, including, for example, amyotrophic lateral sclerosis (ALS), other neuromuscular diseases, Alzheimer's disease, asthma, or the like.

The patient associated with the sensor 142 and/or device 145 may be at a particular COPD stage, exhibiting certain COPD symptoms. For example, the patient may be at an early COPD stage (e.g., stage 1), showing light symptoms and continuing with daily activities; or, the patient may be at a terminal COPD stage (e.g., stage 4), having been recently treated and released from a hospital; or, the patient may be at any other stage of COPD, as will be understood by those skilled in the art. The patient may need to be hospitalized immediately for urgent treatment; or, the patient may be visited on a home-visit basis at a scheduled date/time. The patient may have different healthcare coverages and may be treated by different doctors at different medical facilities. The patient may be provided with the sensor 142, which may detect one or more medical conditions of the patient. The patient may be provided with, or use an existing device 145. The sensor 142 and device 145 may be communicatively linked to each other (e.g., BlueTooth, or other protocol communication link), or provided as a single device (not shown). In this latter regard, the single device may include, for example, a smart watch that monitors biometrics such as oxygen level, heart rate, body temperature, and the like, and communicates a biometric data signal to the server subsystem 120.

The user device subsystem 130 may include one or more mobile devices 132 (e.g., computer, smartphone, tablet, or the like) and/or one or more stationary devices 134 (e.g., computer, or the like). The user associated with the mobile device 132 and/or stationary device 134 may be a health professional, such as, for example, a physician, a physician's assistant, a nurse, an emergency medical technician, a medical administrator, and the like. The user device subsystem 130 may include one or more healthcare facilities, such as, for example, a hospital, a doctor's office, a medical research institute, and the like. The mobile device 132 and/or stationary device 134 may communicate with the server subsystem 120 to retrieve a user database record and generate a user display dashboard that includes user data. The user data may include, for example, patient data for each COPD patient currently being cared for by the user.

The patient database records and the user database records are maintained and updated in the server subsystem 120, in order to have substantially real-time, up-to-date patient information for each COPD patient.

The user may be given access to the user's database record in the server subsystem 120 by, for example, executing a program (e.g., a web-browser, such as Google, FireFox, Safari, or the like) on the device 132 (or 134) and initiating communication with the server subsystem 120 to display a user display dashboard on the device 132 (or 134) (e.g., shown in FIGS. 4A-6F). The dashboard may display patient data, such as, for example, current medical conditions of all patients who are currently under, or will be under the associated user's care, or who have been, or are to be treated by the user; medicines that have been prescribed for each patient; for each patient, whether the patient is developing new COPD symptoms; for each patient, whether each patient's medical conditions are getting worse and require different treatments or immediate hospitalization; each patient's insurance coverage, and for each patient, whether the patient's insurance policy covers the patients' hospital visits, hospitalization, ambulatory services, medical supplies, prescriptions, dietary requirements, meal plans, exercise plans, and the like; for each patient, whether each patient has reached a maximum hospitalization period for a particular time period under the terms set by the insurance policy; and the like. The displayed patient data may include treatment data for each patient, including passive and active treatments.

The server subsystem 120 includes a server 200 and a database 300. The database 300 may be provided in the server 200, or provided separately as seen in FIG. 1. The server 200 may execute the computer program described herein to provide efficient, proactive and thorough COPD patient management services to the users in the user device subsystem 130. The server 200 and the database 300 may be connected to each other and/or the network 110 via the link 105. The server 200 may include, for example, but is not limited to, a computer of an individual, a privately owned entity, a corporation, or the like. The server 200 may initiate communication sessions, or respond to communication request signals from the devices in the user device subsystem 130 and/or the patient device subsystem 140 to establish communication sessions during which the server 200 may send data signals, message signals, control signals, display signals, and the like, with the devices 132, 134, 142 and/or 145. For example, the server 200 may establish a communication session with, or receive a unilateral sensor signal from the sensor 142, wherein the sensor signal may include, for example, a detected medical condition for the associated patient. The server 200 may be connected to the mobile device 132 to send a communication signal to the mobile device, which may include, for example, a patient data, a control signal, a message signal, a display signal, a sound signal, or the like. The patient data may include, for example, a treatment plan, a menu plan, an exercise plan, or the like.

The sensor 142 may be any device that can detect patient condition data, including, for example, a medical condition of the associated patient. As noted previously, the sensor 142 may be a stand-alone sensory device or may be part of a larger system or apparatus. For example, the sensor 142 may include an oxygen level detector for an oxygen tank, which may detect how much oxygen is left in the tank, how much oxygen the patient consumes per use, the patient's blood-oxygen level, and/or the like. The sensor 142 may include a counter that may be part of a medicine dispenser (not shown), which may detect when the patient takes a particular medicine, whether the patient took the prescribed dosage amount of the medicine, whether the patient took the dosage at the prescribed time, and/or the like. The sensor 142 may include a vital sensor, such as, for example, to detect a heartrate or pulse, a respiratory rate, a blood pressure, a blood sugar level, a body temperature, and/or the like, of the patient. The sensor 142 may include a motion detector, which may detect and record movements of the patient. The medical condition detected by the sensor 142 may be sent to the server 200 as a sensor signal via the communication link 105 and network 110.

The server 200 may retrieve a patient database record from the database 300 and update the record with patient condition data received in the sensor signal from the sensor 142 (or device 145). The patient condition data may be received from the sensor 142 (or device 145) periodically (e.g., according to a predetermined schedule), or substantially continuously in near-real-time. The server 200 may communicate with the database 300 and update the corresponding patient database record.

The server 200 may retrieve a user database record from the database 300 and update the patient data in the record. Based on the updated patient data, the server 200 may initiate and send a communication signal to the mobile device 132 and/or stationary device 134 with respect to one or more particular patients under care of the associated user. The user database record may be populated with the updated patient data, or it may include pointer data linking the user database record to each patient database record in the database 300. The server 200 may reference the user database record and initiate and send a Short Message Service (SMS) text signal to the user's smartphone, alerting the user to a particular patient and a particular treatment method.

The server 200 may process the patient data in the database 300 to determine the current medical condition for each patient, update the plan of care for each patient, and the like. The determination may be performed continuously or periodically, such as, for example, by the second, by the minute, hourly, daily, weekly, monthly, quarterly or yearly, or the like.

When the server 200 determines, for example, that a plan of care or a medical condition of a patient, requires immediate medical attention, the server 200 may send a communication signal to the user device 132 (or 134), alerting the associated user of the change in plan of care or medical condition. The communication signal may include an SMS text message, a telephone call, an email, or the like. For example, the server 200 may receive a sensor signal from sensor 142 and search the database 300 to identify the patient database record associated with the sensor signal and/or sensor 142. Based on the patient data in the patient database record, the server 200 may identify the associated user database record and update the user database record with the newly received patient data. The server 200 may then generate and send a communication signal to the device 132 (or 134) associated with the user database record, wherein the communication signal may alert the user of a patient's deteriorating medical condition, failure to follow a prescribed plan of care, or the like.

Alternatively, the sensor 142 may be associated with a user database record, in which case the server 200 may search the database 300, identify the user database record, update the patient data in response to the received sensor signal, generate and send the communication signal to the device 132 (or 134). The server 200 may identify a patient database record based on the patient data in the user database record, retrieve the patient database record from the database 300, update the database record and send the updated patient database record back to the database 300 for storage.

The communication signal sent by the server 200 to the device 132 (or 134) may include detailed information, which the user may use to, for example, proactively contact or visit the patient to check the medical condition, schedule a hospital visit, and/or dispatch an ambulance to the patient. The server 200 may send a communication signal having the same or a similar notification to the device 145 that is associated with the particular patient. Accordingly, the service provider 120 may provide a platform for efficient, thorough, proactive and preemptive COPD patient management services to both the user and the patient.

Each device shown in FIG. 1 may be communicatively coupled to the network 110 and may include a network interface (not shown). The server 200, the databases 300, the mobile device 132, the stationary device 134, the sensor 142, and the device 145 may similarly include a network interface. The network 110 may use a transport layer protocol, such as Transmission Control Protocol (TCP) and/or User Datagram Protocol (UDP), together with a network layer protocol such as Internet Protocol (IP) to transport and manage communication of data packets across the network 110.

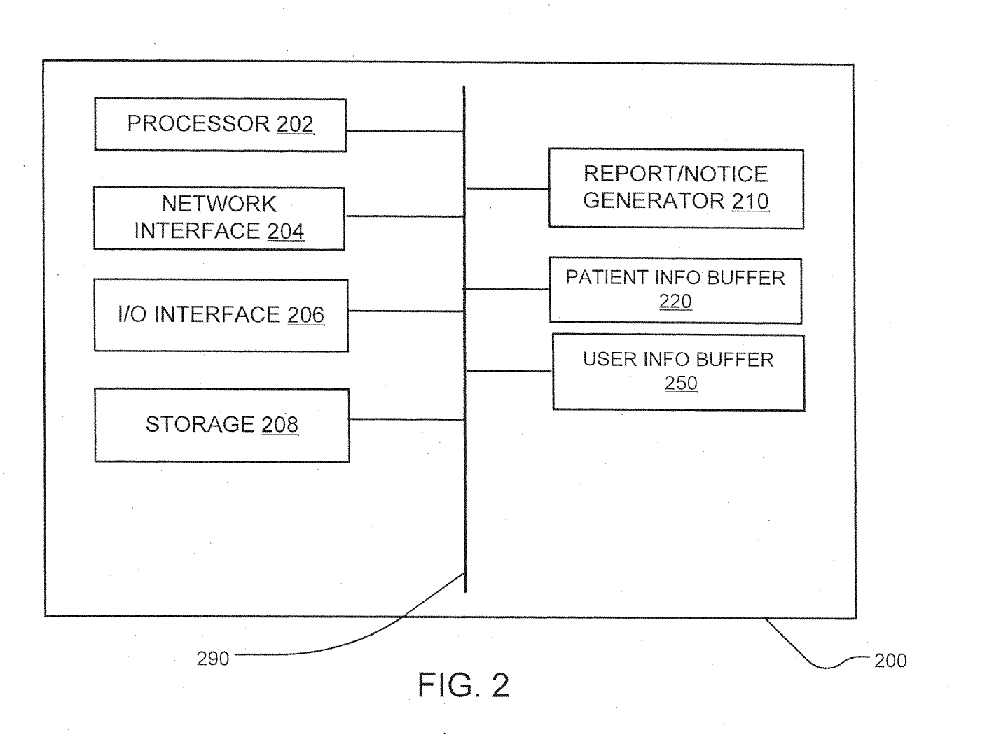

FIG. 2 shows an example of components that may be included in the server 200. The components may be implemented using hardware and/or software. As seen, the server 200 may include a processor 202, a network interface 204, an input/output (I/O) interface 206, a storage 208, a report/notice generator 210, a patient information buffer 220, and a user information buffer, all of which may be connected to a bus 290.

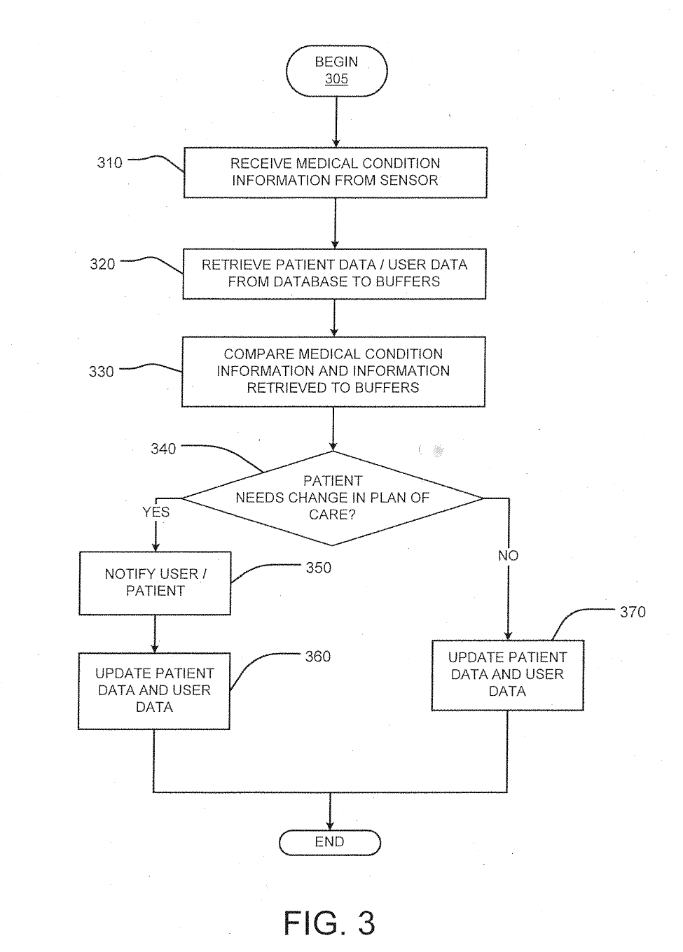

The processor 202 may be configured to execute the processes described herein, including process 305 shown in FIG. 3. The network interface 204 may be configured to initiate, manage and terminate a communication session with the devices connected to the network 110, including the mobile device 132, the stationary device 134, the sensor 142, the device 145, the database 300, and the like. The input/output (I/O) interface 206 may be configured to receive data signals and instruction signals via a user interface (not shown) and output data signals and instruction signals to the same or a different user interface (not shown). The user interface(s) may include a keyboard, keypad, a touchpad, a mouse, a touch-screen display, a display device, a speaker, a microphone, or the like. The storage 208 may include a volatile memory, a non-volatile memory, a random access memory (RAM), a dynamic RAM (DRAM), a static RAM (SRAM), a read-only memory (ROM), a PROM, an EPROM, an EEPROM, or the like.

The patient information buffer 220 may store patient data, which, as noted above, may include, for example, a patient identification (ID) number, a name, an address, a telephone number, healthcare provider information, insurer name, insurer coverage, insurer policy number, current medical conditions, medical history, allergy data, prescription data, IP address, media access control (MAC) address, login identification, password, and the like. The patient data may further include, for example, age, sex, address, emergency contact and the like, of the patient.

The user information buffer 250 may store user data, which, as noted above, may include patient data, or links to patient data for each COPD patient currently being cared for by the user. The user data may include a user's name, telephone number, address, email address, insurance information, qualifications, title or job function (e.g., MD, nurse, nurse's assistant, physical therapist, or the like), work schedule, vacation schedule, and the like. The insurance information may include contact information/preferences of the insurance company, details of the insurance policy, and the like.

The processor 202 may process the patient information stored in the buffer 220 and/or the user information stored in the buffer 250, and store the analysis results data in the storage 208. The processor 202 may forward the analysis results data to the report/notice generator 210, which in turn may generate and send a notification signal to the network interface 204 to include in a communication signal to the device 132 (or 134) and/or the device 145. The report/notice generator 210 may also generate a display signal that presents a user report and/or a patient report, which may be accessed by visiting a website. Non-limiting examples of the web pages for such a web site are shown in FIGS. 4A to 6F, which illustrate an example of a user report that may be displayed on the device 132 (or 134). The displayed report may include different levels of risk for each patient. For instance, the display report may include identification of low risk, medium risk, and/or high risk patients, which may be color-coded (e.g., green, yellow, red), so as to prioritize plans of care based on patient needs. The risk level may be associated with the level of likelihood of hospitalization of the individual based on the current patient data.

FIG. 3 shows an example of a process 300 that may be executed in the network system 100 (shown in FIG. 1) to provide a communication platform that may be used to facilitate managing COPD patients according to the principles of the disclosure. The process 300 may be carried out by the processor 202 of the server 200. The server 200 may include a computer readable medium that comprises a computer program with coded instructions, e.g., computer logic, that, when executed by the server 200, cause the process 300 to be carried out. FIG. 3 (and all other flow diagrams herein) may also represent a block diagram of the components for executing the respective steps. The components of FIG. 3 (as well as the components of any other flow diagram herein) may be software components of computer logic and when the software components are loaded and executed by a computing device, e.g., server 200, may cause the respective action or step of the process to be performed. The components of FIG. 3 (as well as the components of any other flow diagram herein) may reside on a readable tangible or non-transitory storage medium, e.g., a disc or memory, and may comprise a computer readable product.

Referring to FIGS. 1, 2 and 3, the process 300 begins at step 310 by receiving medical condition information in the form of a sensor signal from the sensor 142, which may be stored at the patient info buffer 220. As mentioned above, the medical condition information may include the vital signs (e.g., heartrate or pulse, respiratory rate, blood pressure, blood sugar level, body temperature, blood-oxygen level, etc.), information on whether the patient is taking medications on schedule as prescribed, consuming more painkillers than prescribed, is moving around, and the like. Based on the sensor signal, the processor 202 may query the database 300 at step 320, via the network interface 204, to search and retrieve patient data for the patient data associated with the sensory signal, and temporarily store the patient data in the buffer 220. The processor 202 may compare the received medical condition information with the retrieved patient data at step 330, and update the patient data in the buffer 220 and send the updated patient data to the database 300 for storage.

Based on the patient data associated with the received sensor signal, the processor 202 may query the database 300 to identify and retrieve the user data associated with the patient data (at 320). The processor may temporarily store the user data in the user info buffer 250. The processor may compare the received medical condition information with the patient data at step 330, and update the user data in the buffer 250, and send the updated user data to the database 300 for storage.

At 340, the processor 202 may analyze the comparison data in step 330, including the results of the comparison of the received medical condition information with the patient data and/or user data retrieved from the database 300 to the buffers 220 and 250. For example, the processor 202 may analyze the data comparing the medical condition information with the medical history information stored in the patient information buffer 220 to determine whether a condition or symptom of the patient is getting better or worse. A step 340, a determination is made whether a change in plan of care is necessary based on the received medical condition of the patient. For instance a determination may be made whether the medical condition of the patient has changed beyond a predetermined threshold (e.g., the patient is consuming less oxygen than appropriate, or taking more painkillers than prescribed). The processor 202 may determine that the medical condition of the patient has changed sufficiently to warrant a change in the plan of care for the patient (yes at 340), or that the medical condition does not necessitate a change in the plan of care (no at 340).

The vital signs and other medical indicators may also be used for the comparison at 330. Based on the medical condition information and the comparison result, the processor 202 may determine when the patient needs a change in plan of care, including, for example, medical treatment, hospitalization, ambulance service, medication dosage change, medication change, or the like. For example, the processor 200 may determine a schedule for preemptive medical treatments for the patient when the comparison results indicate that the patient develops new COPD symptoms or the existing symptoms are getting worse. By scheduling the preemptive treatments, the patient may receive timely treatments before developing more severe and terminal COPD symptoms. The comparison results and scheduling information may be stored in the storage 208, and the patient data and/or user data may be updated in the buffers 220 and/or 250, respectively.

If it is determined that a change in plan of care is necessary (yes at 340), the patient and/or user data may be sent to, or retrieved by the report/notice generator 210 at step 350. Depending on the plan of care, the report/notice generator 210 may generate and send a notification signal to the user device 132 (or 134) and/or the patient device 145. The notification signal may include a text message, a telephone call, an email, or the like. The notification signal may include, for example, an instruction to the patient to take a predetermined dosage of a medication at a prescribed time, a notification of a scheduled visit by the user, or the like. The notification signal may include information that the patient's oxygen level is below a prescribed amount (e.g., 65%), the patient has taken too many painkillers, the patient has not moved for a certain period of time, or the like. The report/notice generator 210 may also send a notification to another user device 132 (or 134), such as that of a family member, the primary care physician, a hospital emergency room, or the like, to notify them of the change in plan of care and to take steps that may be necessary in accordance with the updated plan of care (at 350).

Upon receiving such notification, the user of the user device 132 may contact the patient via the user device 145, or his/her family members or guardian via their device 132 (or 134), or dispatch an ambulance to the residence of the patient.

At step 360, the processor 202 may update the user data and/or patient data in the buffers 220, 250, and send the updated data to the database 300 for storage.

If it is determined that a change in plan of care is not necessary (no at 340), then at step 370 the patient and/or user data may be updated to include, for example, the information received with the sensor signal from the sensor 142, including medical condition information. The updated user data and/or patient data may be sent to the database 300 for storage (at 370).

FIGS. 4A-6F show examples of screen images that may be reproduced on the device 132 (or 134) based on a display signal received from the server 200 during a communication session. FIGS. 4A-6F may also represent a block diagram of software components for producing the screen images and receiving input therefrom. The illustrated images may be displayed by the device 132 (or 134) when the device accesses the server 200 over the network 110. The server 200 may receive one or more signals from one or more sensors 142 and/or device(s) 145 indicative of one or more measurements, e.g., oxygen level or carbon dioxide level, taken from a patient under care and tracked by system 100. The server may collate and maintain the one or more measurements from the sensor 142 (or device 145) for one or more patients for displaying on device 132 (or 134) when the device accesses the server 200. The server 200 may track and monitor the activity of the sensor itself to assure it is functioning properly and is maintained according to established policy and schedule.

FIGS. 4A-6F also illustrate various aspects of a user interface that is generated based on the display signal and reproduced on the device 132, after being populated with the user data associated with the user (or user device). The various screen images illustrate an application of the user dashboard for COPD management services, including web pages generated according to the principles of the disclosure.

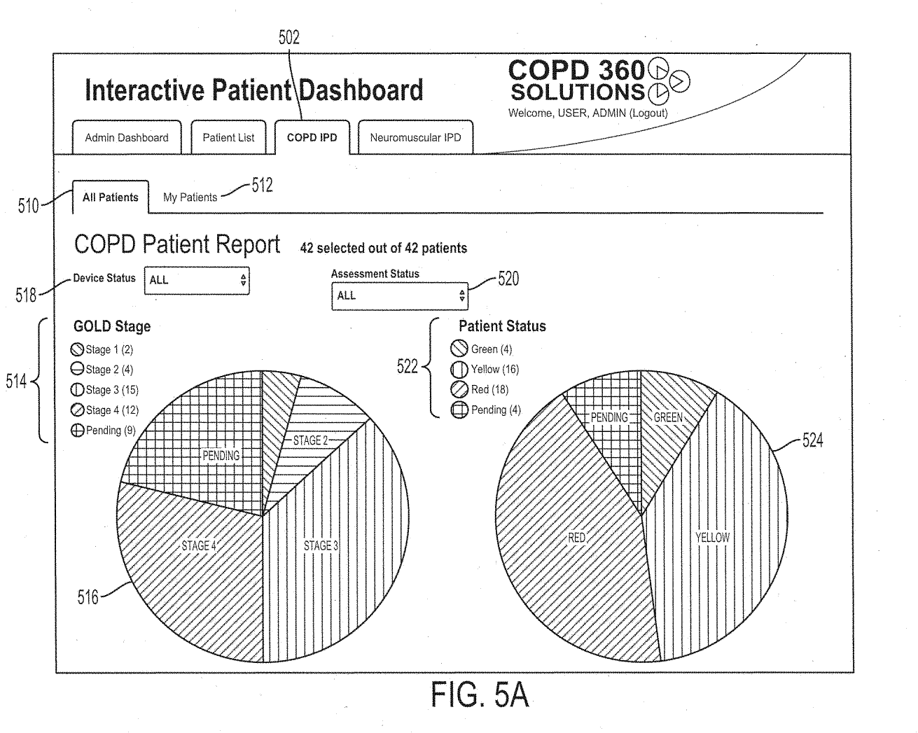

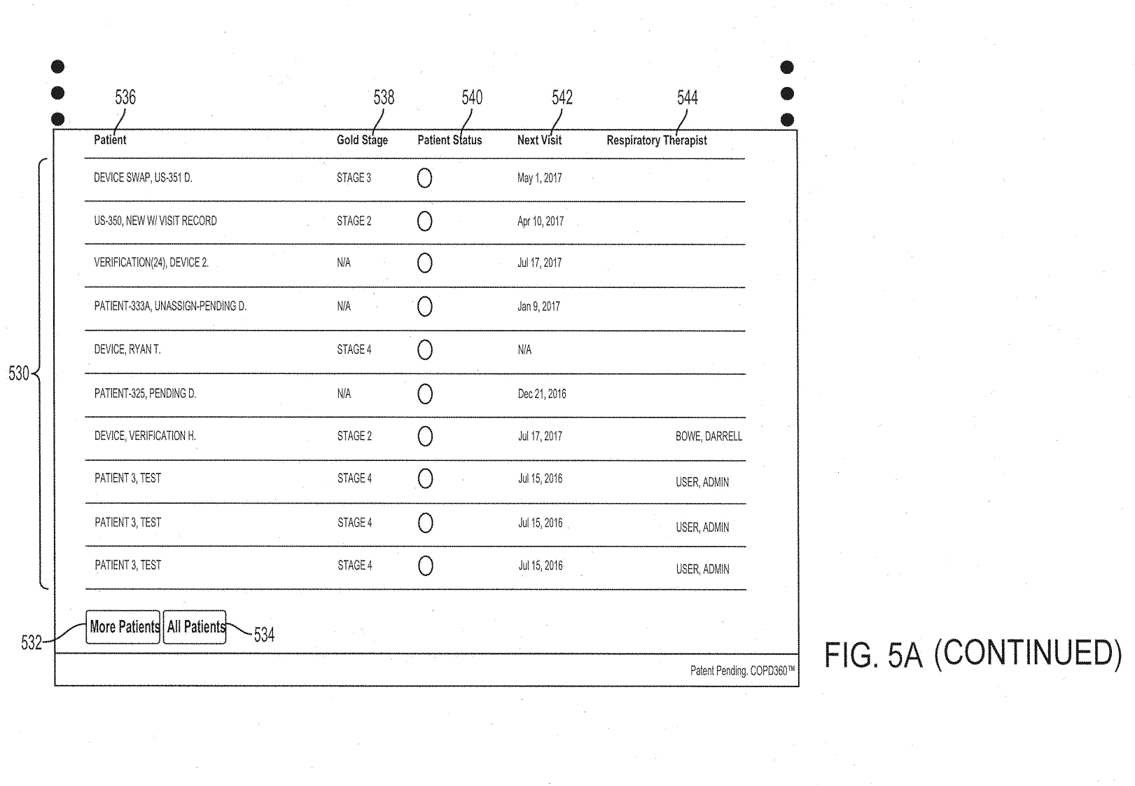

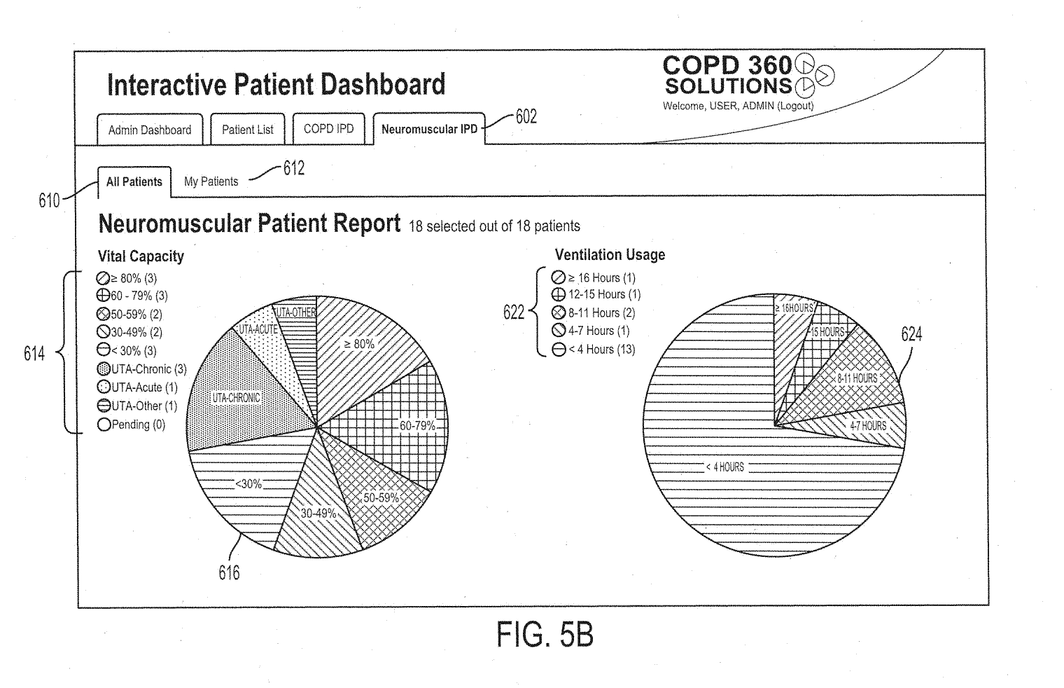

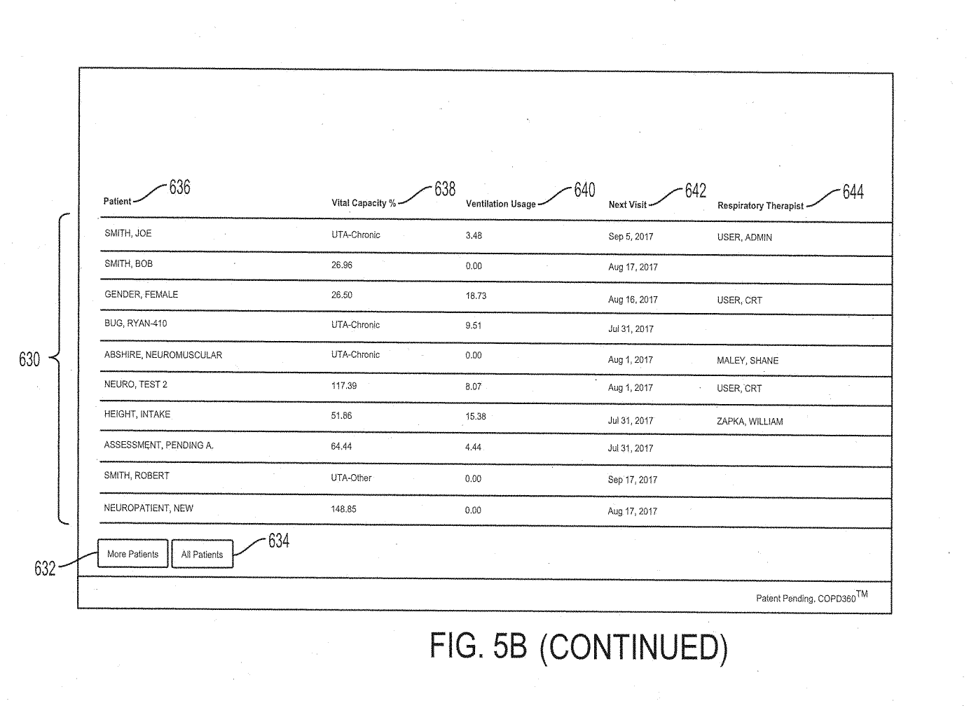

FIGS. 5A and 5B are an example of an interactive patient dashboard (IPD) 400 that may be generated by the server 200 on stationary device 134, mobile device 132, or in some situations device 145. Input may be received via the IPD 400, or subsequently selected IPD pages, e.g., from devices 132 and/or 134, by server 200 for processing. All IPD pages herein may be produced and displayed by server 200. In FIG. 4A, individual patients may be selected 400 by a user via tab 402, for listing all or portions of patients maintained in the system 100. Other tabs COPD 502, Neuromuscular 602 and Admin Dashboard 702 are described more below. A quick select tab 404 may be used by a user to select all or a subset of patients by alphabetic name section for viewing patient information 412. FIG. 4A shows that the ALL Patients tab 406 has been selected causing a display of information 412 of all patients (partial list shown in FIG. 4A). Optionally, a user may select the My Patients tab 408 and patient information associated with the particular user (e.g., a particular health professional that has logged-in) resulting in patient information similar to patient information 412, but limited to those patients associated with the particular user. Patient Visit Queue 410 may be selected for displaying those patients awaiting a healthcare visit or approval of patient record. A Search tab 414 may be used to enter a patient name for a direct search of a patient(s). An Intake New Patient tab 411 may be selected for entering a new patient and patient information into the system 100. An Export Patient Dashboard Report tab 413 may be used to export a patient's information to a report generator or a file. An Initiate TAB 416 may be used to navigate to a screen to enter data for a patient visit. The page may also display a subset of information for patients, including the patient's doctor (e.g., neurologist or pulmonologist), classification (whether the patient is COPD, neuromuscular or other), and an assigned respiratory therapist. Additional metadata shown in this page for each displayed patient may include hospitalization status, device status, next visit data and key health measurements, such as, e.g., vital capacity (i.e., the greatest volume of air that can be expelled from the lungs after taking the deepest possible breath), and forced expiratory volume in the first second (FEV1).

FIG. 4B is an example of a Patient Summary page 442 for a particular example patient named Ackley. This page may be entered, e.g., by selecting the patient name, e.g., Ackley, from FIG. 4A, and Summary 418. This page 442 may show one or more time of visit records and associated data for this patient. A summary portable document format (pdf) or comma-separated values (CSV) export may be created for any visit record by selecting the appropriate button in the array of options 442. Alternatively, all visits may be exported via button 438. Additional visual indicators 421 for each of the visit records 420 may reflect general status of the patient for each visit. For example, the visit dated May 17, 2017 resulted in a “fair” status of the patient as the color of the checkmark to the left of the date May 17, 2017 may be a specific color such as yellow. While the visit of Feb. 8, 2017 indicated an unhealthy status with the “X” status indicator to the left of the date Feb. 8, 2017 being another specific color such as red. If the visit resulted in a good or healthy status, then the indicator for the visit may be another specific color such as green. If no visit was completed, then the status indicator (next to the date) may be another specific color such as blue. In general, the IPD pages herein may contain visual status indicators which may be color coded to quickly convey levels of health for a patient, or for particular parameters for a patient. Tab 422 may be selected to enter or view patient information. Various fields 440 may be selected to export data in CSV or pdf formats, to view record history or to view a full patient report. The various tabs 422, 424, 426, 428, 430, 432, 434, 536 will be discussed in more detail below.

FIGS. 4C and 4D are example visit summary pages displayed when a particular time of visit record is selected from FIG. 4B. In FIG. 4C, the time of visit record dated May 17, 2017 was selected (e.g., clicked-on) from the page shown in FIG. 4B. Status indicator 442 is shown with the same color coding as in FIG. 4B, in this example, yellow. COPD data is shown in FIG. 4C, while neuromuscular data is shown in FIG. 4D. FIG. 4C may include color coded indictors for:
Initial CCQ Score—444
Trending SpO2 Level—446
Currently Smoking—450
CCQ Score—452
Current SpO2 Level—454
Device Compliance—456 (indicates whether the sensor 142 is in compliance with intended usage plan assigned to the patient to monitor if the device is being used properly)
FEV1—458
Weight Change—460
Trending ETCO2 Level—462
Repository Medication Compliance—464
Data submitted—466 (to indicate person creating the record)
Respiratory Therapist—468 (to indicate the patient's therapist name)
Next Visit Date—470
   The data for the indicators in FIG. 4C may be color coded as described above in relation to FIGS. 4A and 4B to give a quick visual feedback to the viewer of the page. For example, the value “FALSE” for Currently Smoking 450 may be color coded green because non-smoking is a good status. Similarly, the value of “3” for Initial CCQ Score may be color coded yellow because a value of “3” may be considered “fair.” Similar color coding for the other values for indictors 446, 448, 452, 454, 456, 458, 460, 462 and 464 may be displayed.

In FIG. 4D, an example of a time of visit record dated Jul. 24, 2017 is shown for a neuromuscular Summary. This example page may be reached in a similar manner as FIG. 4C. The date of visit 442 is shown along with key health measurements for a neuromuscular patient, such as an ALS patient. A visit history tab 474 may be available to select other visit dates. FIG. 4D may include color coded indictors for:
Trending Vital Capacity Sitting Up—472 (breathing capacity of the lungs)
Smoking—474
Trending SpO2 Level—476 (an measurement of oxygen in the blood)
Current Vital Capacity Sitting Up—478
Weight Change—480
Device Compliance—484
Current SpO2 Level—485
Medication Compliance—486 (whether or not the patient is compliant with their medication)
Current end-tidal CO2 (EtCO2) Level—488 (indicates the partial pressure or maximal concentration of CO2 at the end of an exhaled breath, may be expressed as mmHg, or similar units)

The key health measurements shown on the visit summary are consolidated and provide an indication of overall patient health, as summarized below.

### Overview

Overall Status
   Any of the items below that are Red/Unhealthy cause the Red/Unhealthy overall status; the Red/Unhealthy overall status should then be visible on the dashboard.
   If there are no Red/Unhealthy items, any of the items below that are Yellow/Fair cause the Yellow/Fair overall status.
   If there are no Red/Unhealthy or Yellow/Fair items, the overall status will be Green/Healthy.
   If no visit has been completed the overall status will be Blue/Pending
Trending over time:
   From original visit to current visit
      Report Card Elements (Applies to all—COPD, Neuro, Other):
Device Compliance
   Green/Healthy: Operational Hours>or={Hours Required} hours/day—Compliant
   Red/Unhealthy: Operational Hours<{Hours Required} hours/day—Non-compliant
   Compliance Calculation based on comparing A and B:
      A. (Operational Hours Current Visit—Operational Hours Previous Visit)/Number of days between visits
      B. Hours Required per day
If A>=B, then Compliant
If A<B, then Non-Compliant
If there are multiple devices, then compliance is shown for each device. If either device is non-compliant, then the whole row is considered Red/Unhealthy (Note: In a future story, compliance will be based off of a sum of hours of both devices)
If no devices are assigned, the compliance will show N/A
Respiratory Medication Compliance
   Green/Healthy: Compliant (based off of check box on visit medications screen)
   Yellow/Fair: Non-Compliant
Smoking
   Green/Healthy: No
   Yellow/Fair: Yes
Current SpO2 level
   Green/Healthy: >=89%
   Yellow/Fair: 85-88%
   Red/Unhealthy: <85%
Trending SpO2 level
   No health indications—does not affect report card
   Current Visit.SpO2—Initial Visit.SpO2

### COPD-specific Report Card Elements

CCQ Score
   Green/Healthy: 0-2.9
   Yellow/Fair: 3-3.9
   Red/Unhealthy: 4-6
Current EtCO2 level
   No health indications—does not affect report card
   35-45 is good
   46-59 is fair
   60+ is bad
Trending EtCO2 level
   Trend has more meaning than the actual value
   Green/Healthy: <0
   Yellow/Fair: >0-10
   Red/Unhealthy: >10
   Current Visit.EtCO2—Initial Visit.EtCO2 (or comparison of Current Visit to Prior Visit)
FEV1
   No health indications—does not affect report card
   Stage 1—Mild: >=80
   Stage 2—Moderate: >=50-79.99
   Stage 3—Severe: >=30-49.99
   Stage 4—Very Severe <30

### Neuronuscular-Specific Report Card Elements:

Current Vital Capacity % Sitting Up
   Green/Healthy: 60% or more
   Yellow/Fair: 30%-59.99%
   Red/Unhealthy: less than 30%, or NONE (Chronic)
Trending Vital Capacity Sitting Up
   No health indications—does not affect report card
   Current Visit.VC—Initial Visit.VC (or comparison to Prior Visit)
Trending Vital Capacity Percent Sitting Up
   No health indications—does not affect report card
   Current Visit.VCPercent—Initial Visit.VCPercent (or comparison to Prior Visit)

FIG. 4E is an example of a new patient intake screen, configured according to principles of the disclosure. The patient intake screen is used to collect information 480 about a patient when they are entered into the system 100. This information 480 may include their name, contact information, height, weight, date of birth, gender, address, also secondary contact information 480 and medical information 478, any notes 486 and users that have access 487 to this patient. Insurance information 482 and medical provider information 484 may also be recorded. For classification 474 of COPD patients, assignment may be made to a Program 476 to which they are enrolled. Many values related to the patient information, particularly the medical information 478 but others also, of FIG. 4E may also be color coded in a similar manner as data color coded and shown in FIGS. 4A-4D.

FIG. 4F is an example of an evaluation record of a patient, configured according to principles of the disclosure. The evaluation record may be selected by selecting tab 424. The page may include input fields for the following:
Visit date—488
Mental evaluation—490
Physical Evaluation—492
Optionally, note fields for recording sleep habits and diet.
   The values associated with individual fields of FIG. 4F may be color coded similar to FIGS. 4A-4E.

FIG. 4G is an example of a CCQ questionnaire of a patient, configured according to principles of the disclosure. CCQ is one of several questionnaires that could be used to record how a patient is feeling. The clinical CCQ Questionnaire (CCQ) questionnaire may be selected by selecting tab 426 and is used to capture qualitative assessment information 494 related to health and well-being of the patient for accessing the patient's quality of life. Such factors that may be captured may include: shortness of breath at rest, shortness of breath during exercise, concerned about getting cold or breathing becoming worse, depressed because of breathing problems.

FIG. 4H is an example of a device assignment, configured according to principles of the disclosure. One or more devices such as sensors 142 may be assigned to a patient or returned to inventory via devices selection field 496. Detailed information and parameters for the assigned device having an associated serial number may be established in the parametric fields 498. The device shown comprises a ventilator and parameters for its use may be set, which may be recommended manufacturer settings or doctor directed settings. Measurements may be taken at, e.g., a patient's home and delivered to system 100 and server 200 over network 110. Measurements may also be taken during subsequent visits. In this manner, the device compliance in terms of expected usage in hours/day can be determined and tracked.

FIG. 4I is an example of a medications and vaccine tracking page for a patient, configured according to principles of the disclosure. A medication and vaccine information 499 may be captured and entered for storage in system 100. Relevant prescription medication and compliance may be verified with each patient visit. The system 100 may track that a patient has been given one or more vaccines, or scheduled a future date.

FIG. 4J is an example of an exacerbations and hospitalization history for a patient, configured according to principles of the disclosure. The hospitalization history 502 may be maintained by system 100 for each patient as well as exacerbation history 500 for COPD patients. Information related to prior hospitalizations (before current care) 504 may also be captured. Controls 506 for a previous record, discarding changes, saving changes and saving and continuing may be selected.

FIG. 5A is an IPD display that shows the status of all COPD patients, configured according to principles of the disclosure. Using tab 502 to enter this function, FIG. 5A shows a pie chart 516 of the Global Initiative for Obstructive Lung Disease (GOLD) stages 1-4 and pending for all patients in the system 100. (Stages and other important classifications could evolve and IPD can be changed to reflect those movements.) The GOLD stages may be considered a predetermined rating technique. The pie charts 516 and 524 may be color coded for quick assessment of patients' status. Pie chart 524 shows the status of the patients, i.e., green, yellow, red and pending. That is, 18 patients are red condition, 16 patients are yellow condition, 4 patients are green status and 4 patients are pending a status. An assessment status tab may be selected to focus on a particular status. A listing 530 of patients by name 536 showing individual GOLD stage 538, patient status 540 (color coded). and next visit 542 and any assigned respiratory therapist 544 may be displayed. More patients tab 532 and an all patients tab 534 may be selected to control the listing 530.

FIG. 5B is an IPD display that shows the status of all neuromuscular patients, configured according to principles of the disclosure. Using tab 602 to enter this function, FIG. 5B shows a pie chart 616 of vital capacity (percentage) for all patients in the system 100, summarized by key 614. The pie charts 616 and 624 may be color coded for quick assessment of patients' status. Visual pie chart 624 shows the status of the patients by ventilation usage (number of hours) and is summarized by key 622. A listing 630 of patients by name 636 showing individual vital capacity percentage 638, ventilation usage 640 (hours), next visit date 642, and any assigned respiratory therapist 644 may be displayed. More patients tab 632 and an all patients tab 634 may be selected to control the listing 530.

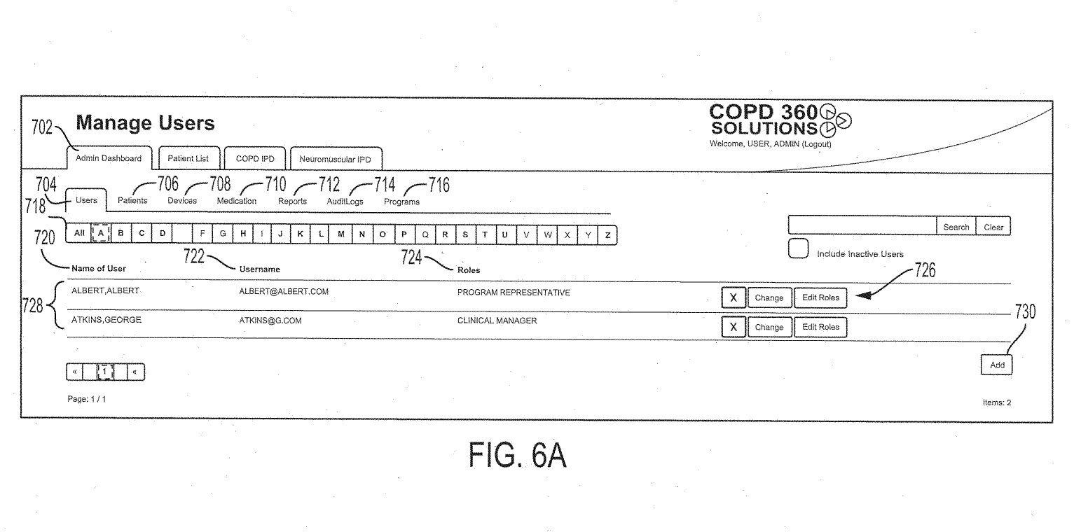

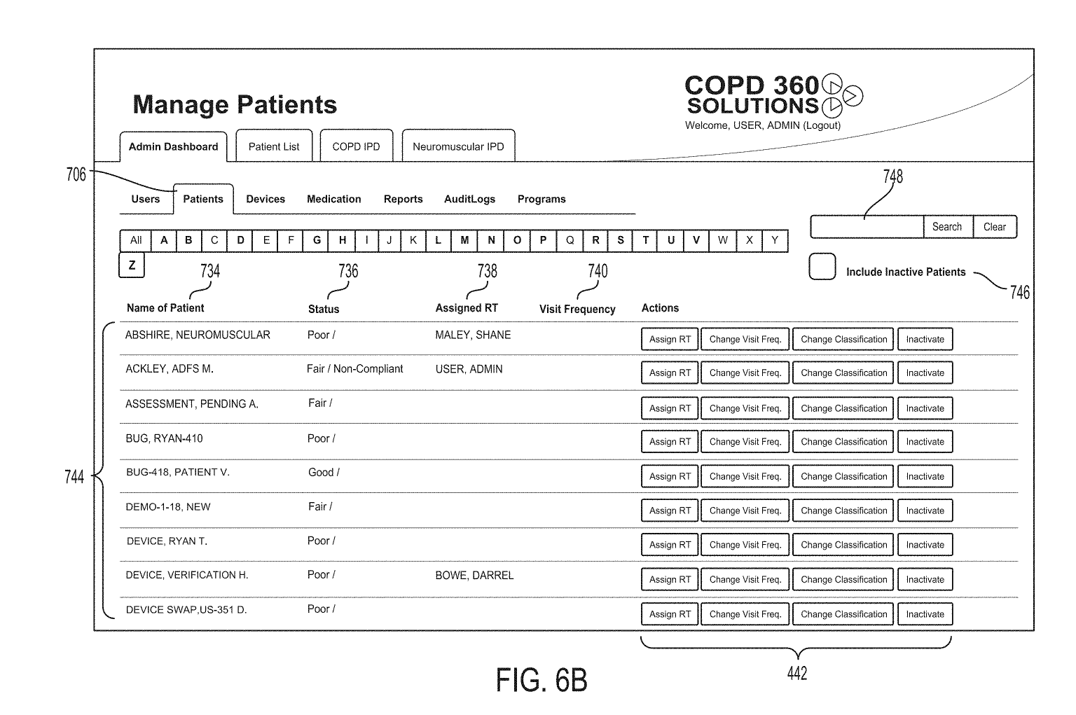

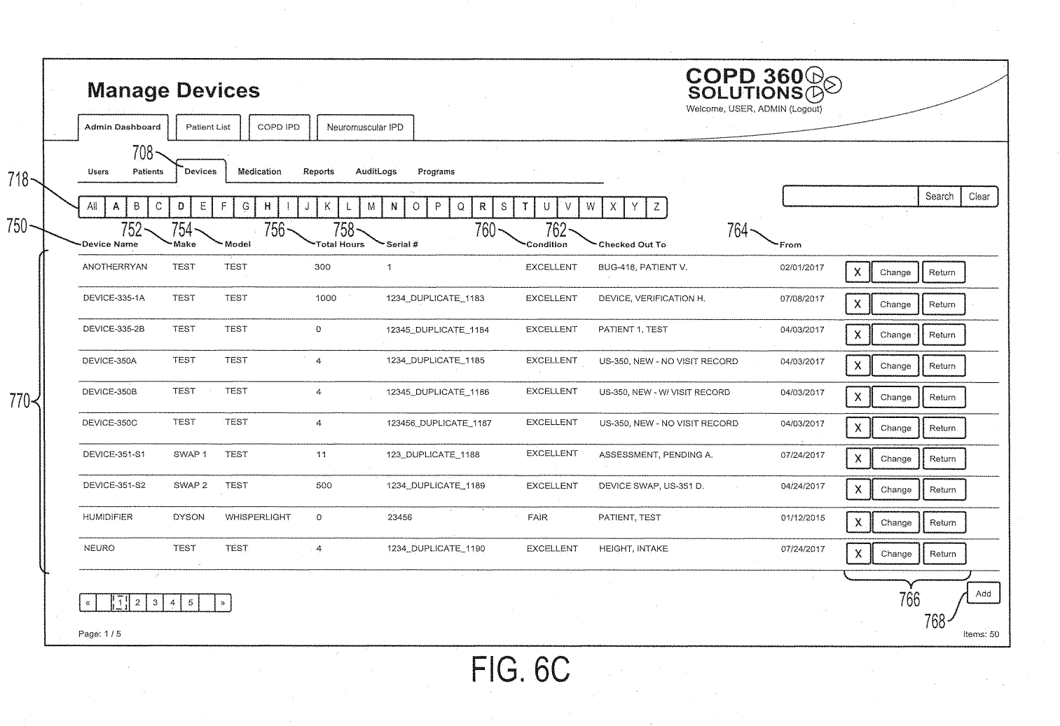

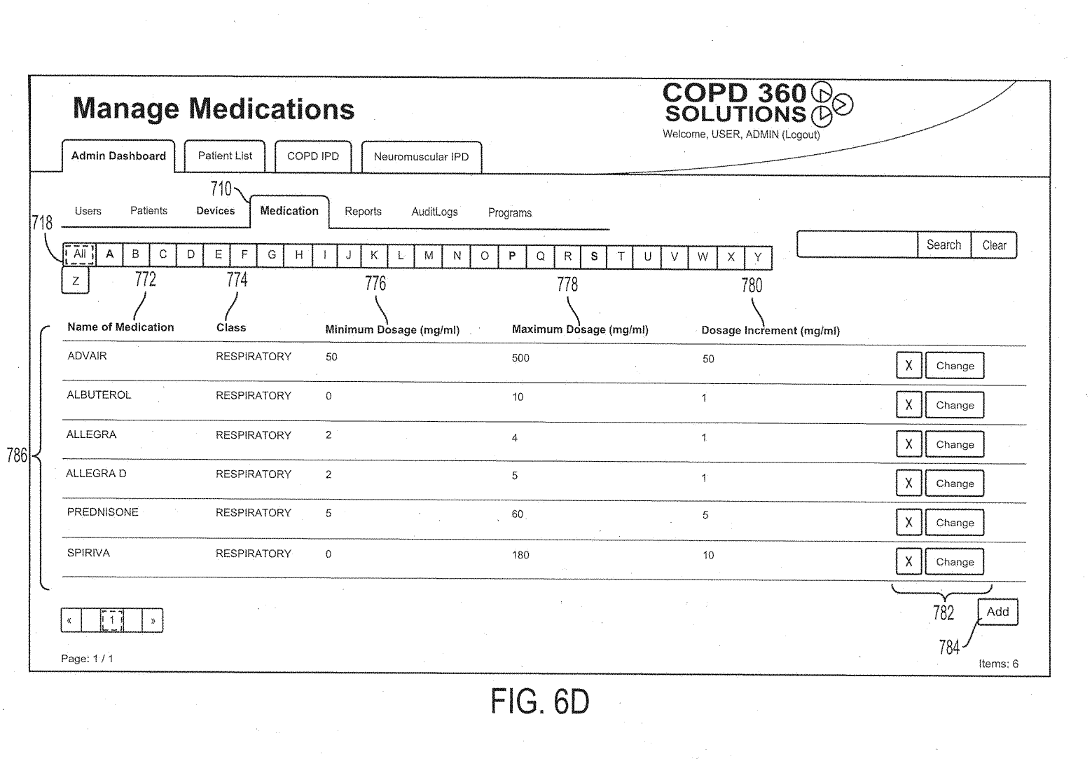

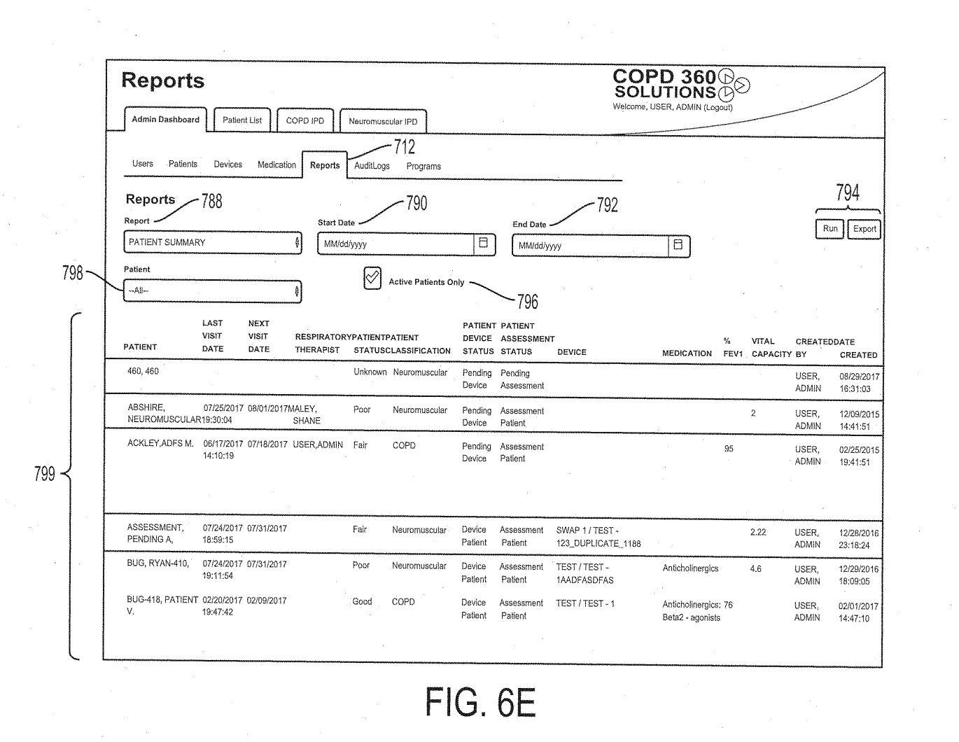

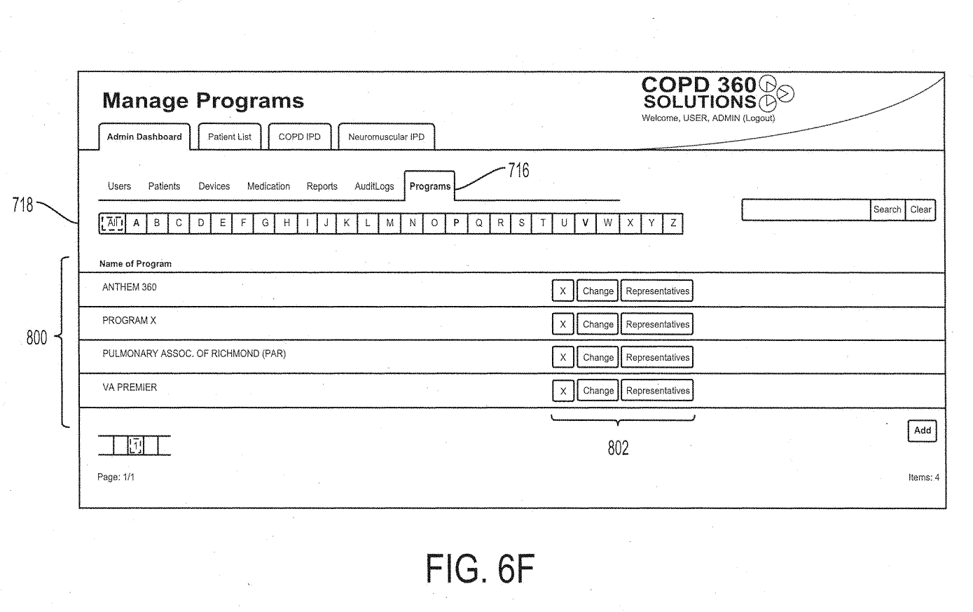

FIG. 6A is an example of an interactive administrative dashboard display, configured according to principles of the disclosure. Entry to the interactive administrative dashboard display may be via tab 702 for managing users access to the system 100. A user such as a respiratory therapist, customer service representative, nurses, doctors, program representatives, and/or system administrators may log into the system 100. Each person or role may have different permissions and access to patient data based on the assigned permissions and access level. A user may select administrating users via tab 704, patients via tab 706, medications via tab 710, generating a report via tab 712, acquiring audit logs 714 of any and all transactions in system 100, and administer programs 716 associated with one or more patients. A navigation bar 718 may be used to navigate to users based on alphabetic names, or to select “all.” In this example, a list 728 including name of user 720 beginning with “A′” may be displayed. Using control tabs 726, a username 722 may be assigned or changed for a user, a role 724 may be assigned or edited or a user may be deleted. Moreover, a new user may be added via tab 730 and subsequent screen (not shown).

FIG. 6B is an example of an interactive administrative dashboard display for managing patients, configured according to principles of the disclosure. Entry to this screen may be via tab 706. A list 744 of patients may be displayed showing their status 736 (poor, fair, good, non-compliant, or the like), assigned respiratory therapist (RT) 738, and visit frequency 740. Changes may be made via control tabs 442, to change visit frequency (e.g., weekly, monthly, biweekly, and the like), assign a RT, change classification of a patient, or to mark a patient inactive. A search for a specific patient may be performed via field 748. An option to include inactive patients in any display may be designated by field 746.

FIG. 6C is an example of an interactive administrative dashboard display for managing devices, configured according to principles of the disclosure. Entry may be made via tab 708. Respiratory and other devices may be logged and maintained in the system 100. The administrator user, or similar person, may easily determine which devices as assigned to which patients and which devices are yet to be assigned. The list 770 of devices may include the device name 750, make 752, model 754, total service hours 756 and serial number 758 may be maintained, as well as the name of the patient 762 and the date 764 the device was assigned. A navigation bar 718 by name of device may be used. Control tabs 766 may be used to change information related to a device, mark the device as returned to stock, or delete the device from the system. A new device may be added to the system 100 via tab 768.

FIG. 6D is an example of an interactive display for managing medications, configured according to principles of the disclosure. A navigation bar 718 may be used to quickly focus on a medication by first alphabetic letter of the name. A list 786 of medications monitored and managed by the system 100 may be displayed including name of the medication 772, class of the medication 774, minimum dosage 776, maximum dosage 778, dosage increment 780. Changes may be made by control tabs 782, and new medications added via tab 784.

FIG. 6E is an example of an interactive display for generating reports, configured according to principles of the disclosure. Entry may be via tab 712. The reporting tool of FIG. 6E may be used by an administrator or other similar user to run reports or create a CSV export file with patient data, such as names, addresses, therapist, classification, and health indicators, their visit history and device status and next visit dates. A type of report 788 may be selected with dates between start date 790 and end date 792. All patients may be selected 798. Only active patients may be selected 796. A list 799 of patients meeting the criteria selected may be displayed or contained in a generated report. Tabs 794 for running a report or exporting a report may be select per the selected parameters.

FIG. 6F is an example of an interactive display for managing programs of related patients, configured according to principles of the disclosure. This tool permits an administrator or other user to change or add a new program that is sponsored by an organization for which patients may be associated. A list 800 of current programs is shown. A navigation bar 718 may be used for quick access by name. A program may be display, modified, changed, or removed, e.g., via tabs 802. A new program may be added also.

The webpages shown in FIGS. 54A-6F may not be accessible if a correct user ID/password combination is not provided. The webpages shown in FIGS. 4A-4E may include four main tabs: Admin Dashboard 702, Patient List 402, COPD IPD 502 and Neuromuscular IPD 602. The system 100 may provide patient and device information or data is available in one place and is available real time to any user with a browser that is authorized to view the data. The information collected may be quite broad, reflecting a holistic approach to patient care and agnostic toward the type of equipment the patient(s) may have in their home. The data can be viewed, trended, analyzed at the individual patient level or for the relevant population (e.g., all patients with COPD or all patients from a specific referral source, or similar criteria). By weighting the various data sets, the software such as executing on server 200 may assess the potential patient risk for an adverse event (e.g., hospitalization). This risk profile can be viewed at the patient or population level. The level of patient risk may be used to determine which patients should get near term attention from a healthcare provider and for what reason. By targeting patients by risk profile, the system 100 software may drive an efficient use of healthcare providers (e.g., informed resources visiting the riskiest patients). Embedded intelligence via the system 100 software may improve and standardize care at the Point of Care (e.g., the patient's home). Using cues and prompts (the Respiratory Therapists, for example) can help facilitate improved clinical care for the patients. By collecting critical clinical data in a longitudinal record, it gives us the ability to analyze the information for best practices, correlations, and the like. The software tools provided by system 100 also may be used to evaluate Respiratory Therapist performance based on risk adjusted patient results. All of the structured data maintained in system 100 may be available in reports for use by healthcare providers, administrators, referral sources, or others with the right and interest to view system data. The server 200 may also provide a tool, e.g. generator 210, that calculates and compares data for evaluating Respiratory Therapist performance, quality and consistency, based at least in part on collected patient data and historical records. For example, performance of one therapist may be compared with another therapist based on metrics generated by server 200 based on historical data, timeliness, patient visits and patient data stored in the database. Data produced and displayable by the server may be searchable and reportable. This may include the structured data displayed or received, e.g., data as shown in conjunction with FIGS. 4A-6F.

A “computer” and/or a “device” as used in this disclosure, means any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a super computer, a personal computer, a laptop computer, a palmtop computer, a notebook computer, a desktop computer, a workstation computer, a server, a cloud of servers, a server farm, a smart phone, a smart television, a smart appliance, or the like, or an array of processors, microprocessors, central processing units, general purpose computers, super computers, personal computers, laptop computers, palmtop computers, notebook computers, desktop computers, workstation computers, servers, smart phones, smart televisions, smart appliances, or the like.

A “server,” as used in this disclosure, means any combination of software and/or hardware, including at least one application and/or at least one computer to perform services for connected clients as part of a client-server architecture. The at least one server application may include, but is not limited to, for example, an application program that can accept connections to service requests from clients by sending back responses to the clients. The server may be configured to run the at least one application, often under heavy workloads, unattended, for extended periods of time with minimal human direction. The server may include a plurality of computers configured, with the at least one application being divided among the computers depending upon the workload. For example, under light loading, the at least one application can run on a single computer. However, under heavy loading, multiple computers may be required to run the at least one application. The server, or any if its computers, may also be used as a workstation.

A “database,” as used in this disclosure, means any combination of software and/or hardware, including at least one application and/or at least one computer. The database may include a structured collection of records or data organized according to a database model, such as, for example, but not limited to at least one of a relational model, a hierarchical model, a network model or the like. The database may include a database management system application (DBMS) as is known in the art. The at least one application may include, but is not limited to, for example, an application program that can accept connections to service requests from clients by sending back responses to the clients. The database may be configured to run the at least one application, often under heavy workloads, unattended, for extended periods of time with minimal human direction.

A “communication link,” as used in this disclosure, means a wired and/or wireless medium that conveys data or information between at least two points. The wired or wireless medium may include, for example, a metallic conductor link, a radio frequency (RF) communication link, an Infrared (IR) communication link, an optical communication link, or the like, without limitation. The RF communication link may include, for example, WiFi, WiMAX, IEEE 802.11, DECT, 0G, 1G, 2G, 3G or 4G cellular standards, Bluetooth, and the like.

A “network,” as used in this disclosure means, but is not limited to, for example, at least one of a local area network (LAN), a wide area network (WAN), a metropolitan area network (MAN), a personal area network (PAN), a campus area network, a corporate area network, a global area network (GAN), a broadband area network (BAN), a cellular network, a Peer-to-Peer (“P2P”) network (e.g., BitTorrent, the Internet, or the like, or any combination of the foregoing, any of which may be configured to communicate data via a wireless and/or a wired communication medium. These networks may run a variety of protocols not limited to TCP/IP, IRC or HTTP.

The terms “including,” “comprising” and variations thereof, as used in this disclosure, mean “including, but not limited to,” unless expressly specified otherwise.

The terms “a,” “an,” and “the,” as used in this disclosure, means “one or more,” unless expressly specified otherwise.

Devices that are in communication with each other need not be in continuous communication with each other, unless expressly specified otherwise. In addition, devices that are in communication with each other may communicate directly or indirectly through one or more intermediaries.

Although process steps, method steps, algorithms, or the like, may be described in a sequential order, such processes, methods and algorithms may be configured to work in alternate orders. In other words, any sequence or order of steps that may be described does not necessarily indicate a requirement that the steps be performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

A “computer-readable medium,” as used in this disclosure, means any medium that participates in providing data (for example, instructions) which may be read by a computer. Such a medium may take many forms, including non-volatile media, volatile media, and transmission media. Non-volatile media may include, for example, optical or magnetic disks and other persistent memory. Volatile media may include dynamic random access memory (DRAM). Transmission media may include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, non-transitory mediums, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium may include a “Cloud,” which includes a distribution of files across multiple (e.g., thousands of) memory caches on multiple (e.g., thousands of) computers.

Various forms of computer readable media may be involved in carrying sequences of instructions to a computer. For example, sequences of instruction (i) may be delivered from a RAM to a processor, (ii) may be carried over a wireless transmission medium, and/or (iii) may be formatted according to numerous formats, standards or protocols, including, for example, WiFi, WiMAX, IEEE 802.11, DECT, 0G, 1G, 2G, 3G or 4G cellular standards, Bluetooth, or the like.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications, or modifications of the disclosure.

What is claimed is:

1. A network system for managing patient care, comprising:
a server operatively in communication with at least one health sensor over a network, the at least one health sensor assigned to at least one patient, the server configured to receive at least one message from the at least one health sensor containing data associated with sensed patient data;
a database coupled to the server for storing the sensed patient data, and
displaying on a display device by the server a status of at least one health condition of the patient according to a predetermined rating and based on a history of the at least one received messages stored in the database for managing treatment of the at least one patient.

2. The network system of claim 1, wherein the at least one health sensor measures oxygen levels or carbon dioxide levels in the at least one patient.

3. The network system of claim 1, wherein the at least one health sensor comprises a plurality of sensors and the at least one message is a plurality of messages received by the server from different sensors of the plurality of sensors over a period of time.

4. The network system of claim 1, wherein the health condition is a Chronic Obstructive Pulmonary Disease (COPD) or a neuromuscular disorder.

5. The network system of claim 4, wherein the server is configured to display a list of patients related to COPD or the neuromuscular disorder.

6. The network system of claim 4, wherein the server is configured to display a historical summary of patient visits including at least one color coded visual indicator associated with each displayed patient visit, the at least one color coded visual indicator indicative of a specific health characteristic of the patient.

7. The network system of claim 1, wherein the server is configured to display a summary page of an individual COPD patient that includes a plurality of, in any combination:
a) an initial CCQ Score,
b) a trending SpO2 Level,
c) current ETCO2 Level,
d) a currently smoking Indicator,
e) a CCQ score,
f) a current SpO2 level,
g) a device compliance indicator,
h) a weight change indicator,
g) a trending ETCO2 Level,
h) a respiratory medication compliance indicator,
i) a respiratory therapist assigned to the patient, and
k) a separate visual color coded indicator indicative of a specific health characteristic of the COPD patient.

8. The network system of claim 7, wherein any indicator or level is itself color coded to indicate a level of health or compliance.

9. The network system of claim 1, wherein the server is configured to display a summary page of an individual neuromuscular patient that includes a plurality of, in any combination:
a) trending vital capacity sitting up indicator,
b) a smoking indicator,
c) a trending SpO2 level indicator,
d) a current vital capacity sitting up indicator,
e) a weight change indicator,
f) a device compliance indicator,
g) a current SpO2 level,
h) a medical compliance indicator,
g) a current EtCO2 Level,
h) a respiratory therapist, and
i) a separate color coded visual indicator indicating a specific health characteristic of the patient.

10. The network system of claim 10, wherein any indicator or level is itself color coded to indicate a level of health or compliance.

11. The network system of claim 5, wherein the server is configured to display a patient intake form and receiving input date for adding a new patient to the network system.

12. The network system of claim 5, wherein the server is configured to display a patient evaluation form for inputting results of a physical evaluation and a mental evaluation.

13. The network system of claim 5, wherein the server is configured to display a CCQ questionnaire for receiving input.

14. The network system of claim 5, wherein the server is configured to display a device management screen to enter information relate to a new device including a health sensor into system inventory, remove a device from system inventory, or to assign a device to a patient, the information including a serial number, model and manufacturer.

16. The network system of claim 5, wherein the server is configured to display a medication and vaccine screen for adding, updating or removing a medication or vaccine in the system, or for noting potential medication variances from standard practice.

17. The network system of claim 5, wherein the server is configured to display a COPD interactive patient dashboard that includes an indication of all or a portion of patients managed by the system by individual Global Initiative for Obstructive Lung Disease (GOLD) stages.

18. The network system of claim 17, wherein the indication is a color coded pie chart.

19. The network system of claim 5, wherein the server is configured to display a neuromuscular Interactive patient dashboard that includes an indication of all or a portion of patients managed by the system by vital capacity or ventilation usage.

20. The network system of claim 19, wherein the indication is a color coded pie chart.

21. A method for managing patient care, comprising:
receiving at a server over a network from at least one health sensor assigned to at least one patient, at least one message from the health sensor containing data associated with sensed patient data;
capturing the sensed patient data and storing in a database; and
displaying on a display device by the server a status of at least one health condition of the patient according to a predetermined rating based on a history of the at least one received messages stored in the database for managing treatment of the at least one patient.

22. The method of claim 21, wherein the at least one health sensor measures oxygen levels or carbon dioxide levels in the at least one patient.

23. The method of claim 21, wherein the at least one health sensor comprises a plurality of sensors and the at least one message is a plurality of messages received by the server from different sensors of the plurality of sensors over a period of time.

24. The method of claim 21, wherein the health condition is a Chronic Obstructive Pulmonary Disease (COPD) or a neuromuscular disorder.

25. The method of claim 21, further comprising display a summary page of an individual COPD patient that includes a plurality of, in any combination:
a) an initial CCQ Score,
b) a trending SpO2 Level,
c) current ETCO2 Level,
d) a currently smoking Indicator,
e) a CCQ score,
f) a current SpO2 level,
g) a device compliance indicator,
h) a weight change indicator,
g) a trending ETCO2 Level,
h) a respiratory medication compliance indicator,
i) a respiratory therapist assigned to the patient, and
k) a separate visual color coded indicator indicative of a general current overall health of the COPD patient,
wherein any indicator or level is itself color coded to indicate a level of health or compliance.

26. The method of claim 21, further comprising displaying a summary page of an individual neuromuscular patient that includes a plurality of, in any combination:
a) trending vital capacity sitting up indicator,
b) a smoking indicator,
c) a trending SpO2 level indicator,
d) a current vital capacity sitting up indicator,
e) a weight change indicator,
f) a device compliance indicator,
g) a current SpO2 level,
h) a medical compliance indicator,
g) a current EtCO2 Level,
h) a respiratory therapist, and
i) a separate color coded visual indicator indicating a general overall health of the patient,
wherein any indicator or level is itself color coded to indicate a level of health or compliance.

27. A computer program product embodied on a non-transitory computer-readable medium that comprise computer logic that when read and executed by a computer performs the following:
receiving at a server over a network from at least one health sensor, including an oxygen sensor or carbon dioxide sensor, assigned to at least one patient, a plurality of messages from the health sensor containing data associated with sensed patient data;
capturing the sensed patient data and storing in a database; and
displaying on a display device by the server a status of at least one health condition including a Chronic Obstructive Pulmonary Disease (COPD) or a neuromuscular disorder of the patient according to a predetermined rating based on a history of the plurality of received messages stored in the database for managing treatment of the at least one patient.

28. The computer program product of claim 27, wherein all patient data stored in the database, or data produced and displayable by the server, is searchable and reportable.

29. The computer program product of claim 28, wherein the server provides a tool for evaluating Respiratory Therapist performance, quality and consistency based on metrics related to data stored in the database.
